(19) United States
(12) Patent Application Publication
(10) Pub. No.: US 2026/0056138 A1
(43) Pub. Date: Feb. 26, 2026

(54) Methods Of Using Harmonic Transponders To Measure Environmental Conditions, And Related Methods, Systems, And Software

(71) Applicant: University of Vermont and State Agricultural College, Burlington, VT (US)

(72) Inventor: Jeffrey Frolik, Essex Junction, VT (US)

(21) Appl. No.: 19/355,340

(22) Filed: Oct. 10, 2025

Related U.S. Application Data
(63) Continuation-in-part of application No. PCT/US2024/025036, filed on Apr. 17, 2024.
(60) Provisional application No. 63/497,113, filed on Apr. 19, 2023.
(60) Provisional application No. 63/497,179, filed on Apr. 19, 2023.
(60) Provisional application No. 63/618,488, filed on Jan. 8, 2024.

(51) Int. Cl. G01N 22/04; G01N 33/24; G01N 33/38

(52) U.S. Cl. CPC G01N 22/04; G01N 33/241; G01N 33/383

(57) ABSTRACT

Aspects of the present disclosure include systems and methods for determining the loss of an unknown channel through remote wireless interrogation of a nonlinear transponder. The transponder's nonlinearity is leveraged to ascertain an operating point of the nonlinear transponder. In some examples the operating point is determined by interrogating a nonlinear transponder with an interrogation signal containing two closely spaced frequencies and measuring intermodulation distortion and/or other components of a return signal that nonlinear transponder backscatters. In some examples, an operating point is determined by interrogating the transponder with an amplitude-modulated interrogation signal, comprising a carrier frequency modulated with at least one second frequency and measuring components of a return signal backscattered by the nonlinear transponder. With an operating point determined, channel losses and material properties of an environment in which the transponder is located, among other things, can be determined.

### RELATED APPLICATION DATA

This application is a bypass continuation-in-part of International Patent Application PCT/US2024/025036, filed on Apr. 17, 2024, and titled “Methods Of Sensing Physical Characteristics Of Medium Using Backscattered Signals From Embedded Nonlinear Transponders, And Related Systems And Software”, which claims the benefit of priority of U.S. Provisional Patent Application Ser. No. 63/497,113, filed on Apr. 19, 2023, and titled “System And Method To Remotely Determine Channel Loss Using Embedded Non-Linear Transponders”. This application also claims the benefit of priority of U.S. Provisional Patent Application Ser. No. 63/497,179, filed on Apr. 19, 2023, and titled “Method And System For Determining Material Content”. This application further claims the benefit of priority of U.S. Provisional Patent Application Ser. No. 63/618,488, filed on Jan. 8, 2024, and titled “Ascertaining Operating Points of Harmonic Transponders Using Intermodulation Responses and Measuring Media Absorption Loss Using Embedded Harmonic Transponders”. Each of the foregoing referenced applications is incorporated by reference into this application in its entirety.

### STATEMENT OF GOVERNMENT INTEREST

This invention was made with government support under grant 693JJ620C000004 awarded by the U.S. Department of Transportation and grant 2023-67022-38830 awarded by the National Institute of Food and Agriculture of the U.S. Department of Agriculture. The government has certain rights in the invention.

### FIELD OF THE DISCLOSURE

The present disclosure generally relates to the field of nonlinear transponders. In particular, the present disclosure is directed to methods of using harmonic transponders to measuring environmental conditions, and related methods, systems, and software.

### BACKGROUND

Wireless sensors are a means by which we can better monitor, and thus better understand, both natural and built environments. For many applications, sensors may need to be deployed for extended periods of time (e.g., days, months, or years) in environments where battery replacement is not tenable (e.g., within soil, concrete, or other medium) and/or at locations where it is impractical or otherwise prohibitive to install active devices, such as transmitters, receivers, signal analyzers, etc., needed to achieve a desired goal, such as characterizing wireless communication channels in difficult environments. For such applications, passive wireless sensors are a promising solution.

Harmonic transponders are one such wireless passive device, and which have been investigated for a variety of novel sensing purposes, including tracking of insects, locating buried infrastructure, monitoring temperature and humidity, and ascertaining soil moisture. Harmonic transponders receive a signal from an interrogator, typically, at a single frequency, f₀. As illustrated in FIG. 1—Top, this signal is rectified using a diode. By way of example, in the case of a pure sinusoid input (interrogation signal) with a single fixed frequency that has a certain period in time over which the shape repeats, the output from the diode is the rectified sinusoid, e.g., only the positive portion of the signal, that also repeats with the same period. Rectification is a nonlinear operation and as a result, the signal reradiated (i.e., backscattered) to the interrogator consists of harmonics of the input frequency. Often, the return signal of interest is in the band of the second harmonic of the interrogation signal, i.e., 2×f₀, which is the case for some of the example implementations described herein. The tuning stubs and inductors shown in FIG. 1 are used to set the center frequency for the transponder's operation. That is, the frequency at which it can be expected to produce the strongest return.

For some of the above applications (e.g., temperature and humidity) the parameter of interest can be measured at the point at which the device is located. However for others (e.g., soil moisture, snow water content, multipath characteristics of wireless channels), the parameter of interest is captured by a volume. One way to determine soil moisture and multipath characteristics is to measure the propagation loss seen in the forward and/or reverse links, that is, the change in the power in the propagating signal as it travels to the harmonic transponder from the interrogator (forward link) and back (reverse link) (see FIG. 1). However, as described herein, isolating these losses from the overall bidirectional link loss, which also includes the nonlinear harmonic transponder, is not a straightforward task.

### SUMMARY OF THE DISCLOSURE

In one implementation, the present disclosure is directed to a method of analyzing an environmental condition. The method includes wirelessly transmitting an interrogation signal that contains first and second frequencies to a nonlinear frequency-multiplying transponder; receiving a backscattered signal from the nonlinear frequency-multiplying transponder, wherein the backscattered signal contains multiple loss-characterizing components; measuring features of the multiple loss-characterizing components; and determining, using the measured features, one or more loss indicia that correlate to environmental losses between the transmitting of the interrogation signal and the receiving of the backscattered signal.

In another implementation, the present disclosure is directed to machine-readable storage medium containing machine-executable instructions for performing the method described immediately above.

### BRIEF DESCRIPTION OF THE DRAWINGS

For the purpose of illustration, the drawings show aspects of one or more embodiments of this disclosure. However, it should be understood that the present disclosure is not limited to the precise arrangements and instrumentalities shown in the drawings, wherein:

FIG. 1A is a diagram illustrating an example nonlinear transponder of a second-harmonic (2H) type;

FIG. 1B is a high-level diagram illustrating an example sensor system having a 2H harmonic transponder embedded in a medium;

FIG. 2 is a diagram illustrating nomenclature used in an example method of determining an operating point (OP) of a 2H transponder;

FIG. 3 is a diagram illustrating a method of interrogating a 2H transponder using two-frequency interrogation signal and determining an OP of the 2H transponder using components within the second-harmonics band of the signal backscattered from the 2H transponder;

FIG. 4 is a diagram illustrating components of a signal backscattered from an interrogation signal containing a first frequency, f₁, of 886.65 MHz and a second frequency, f₂, of 886.85 MHz incident a 2H transponder and measured at second harmonics (2H=1773.3 MHz and 1775.7 MHz), a second-order intermodulation distortion product (IM2=1773.5 MHz), and fourth-order (IM4=1773.1 MHz and 1773.9 MHz), with incident power at normally oriented (φ=0) transponder and an OP=−27 dBm;

FIG. 5 is a plot of conversion loss of the 2H transponder of FIG. 4 as a function of the power incident to the 2H transponder (a/k/a, the OP), plotted along with the ratio of the second-order IMD (IM2) component to the mean of the second harmonics and to the mean of the fourth order IMD (IM4) components;

FIG. 6A is a plot of the OP (lefthand y-axis) and the conversion loss (righthand y-axis) of a 2H transponder as a function of a ratio of the IM2 component of FIG. 4 to a second harmonic of FIG. 4;

FIG. 6B is a plot of the OP (lefthand y-axis) and the conversion loss (righthand y-axis) of the 2H transponder of FIG. 6A as a function of a ratio of the IM2 component of FIG. 4 to the IM4 component of FIG. 4;

FIG. 7 shows an example amplitude-modulation (AM) interrogation signal, wherein a carrier of at 900.000 MHz is tone modulated with a 1 kHz tone using a modulation index, μ, of 100%;

FIG. 8 is a plot of conversion loss versus the operating point of an example 2H transponder, showing the minimum conversion loss occurring at an incident power at the 2H transponder of −25 dBm;

FIG. 9 is a plot of a carrier to sideband ((SB) ratio versus the OP of an example 2H transponder, with horizontal lines indicated ratio at the transmitting source for amplitude modulation constants μ=25%, 50%, 75%, and 100%;

FIG. 10 is a plot of conversion loss data of a harmonic transponder buried under 40 cm snowpack using a dual-frequency interrogation approach;

FIG. 11A is a plot of forward-link loss measurements using an AM interrogation approach, illustrating the transmitted power needed to reach the 12 dB C/SB in the cases of snow and no snow;

FIG. 11B is a plot of reverse-link loss measurements using an AM interrogation approach, illustrating the decrease in received power when the device was buried under snow, relative to the no snow case;

FIG. 12 is a plot of normalized conversion loss (CL) data relative to the inflection power point (IPP) for each of a harmonic transponder having a planar design and a harmonic transponder having a 3D design;

FIG. 13 is a diagram of a test setup used to test harmonic transponders to investigate soil-moisture testing using a constant-tone modulated AM interrogation approach;

FIG. 14A is a plot of C/SB versus incident power normalized to the IPP of the harmonic transponder of the planar design, wherein matched power points (MPPs) are shown by circles;

FIG. 14B is a plot of C/SB versus incident power normalized to the IPP of the harmonic transponder of the 3D design, wherein MPPs are shown by circles;

FIG. 15 is a plot of C/SB versus incident power for the planar harmonic transponder represented in FIG. 14A, showing forward link losses of 3.25 dB (a), 4.0 dB (b), 10.25 dB (c), and 18.25 dB (d) for differing soil conditions relative to no soil, specifically dry, and 10%, 20%, and 25% soil moisture content, respectively;

FIG. 16 is a plot illustrating a response of a commercial off-the-shelf harmonic transponder, buried under a snowpack, to a AM interrogation signal of the present disclosure, wherein the 30-cm data was adjusted based on measurement offsets seen across frequencies at that depth;

FIG. 17 is a diagram of a test setup for mapping antenna performance of a 2H transponder using a dual-frequency-interrogation approach;

FIG. 18 is a plot of conversion loss (left axis) and component ratios (right axis) for the 2H transponder of FIG. 17;

FIG. 19 is a plot of total conversion loss of the 2H transponder of FIG. 17 over azimuth data decomposed by the relative contribution of the transponder's receive antenna gain pattern, transmit antenna gain pattern, and diode conversion loss;

FIG. 20 is a flow diagram of an example method interrogating a 2H transponder using a dual-spaced-apart frequency approach and processing the returned signal to determine one or more characteristics of the returned signal;

FIG. 21 is a flow diagram of an example method of interrogating a 2H transponder using a tone-modulated AM signal approach and processing the returned signal to determine one or more characteristics of the returned signal;

FIG. 22 is a flow diagram of an example method of determining properties of a medium using dual-spaced-apart frequency interrogation of a 2H transponder embedded in the medium;

FIG. 23 is a flow diagram of an example method of determining properties of a medium using tone-modulated AM interrogation of a 2H transponder embedded in the medium;

FIG. 24 is a high-level block diagram of an example interrogator made in accordance with the present disclosure and configured to perform dual-spaced-apart frequency methods disclosed herein;

FIG. 25 is a high-level block diagram of an example interrogator made in accordance with the present disclosure and configured to perform tone-modulated AM methods disclosed herein;

FIG. 26 is high-level block diagram of an example interrogator made in accordance with the present disclosure, illustrating how certain functions can be implemented by a software defined radio;

FIG. 27 is a diagram illustrating a conventional channel-sounding setup for characterizing multipath of a wireless link in a multipath environment, wherein the setup requires active instrumentation at both ends of the wireless link;

FIG. 28 is a diagram of an example channel-sounding setup of the present disclosure for characterizing multipath of a wireless link in a multipath environment, wherein the setup has active instrumentation at one end of the wireless link and a passive harmonic transponder at the other end;

FIG. 29 is a combined graph comparing single carrier conversion loss ((L) (solid line) to carrier-to-sideband values ((SB) (dashed line) of a spatially-dependent fading example, with the CL and C/SB values measured when interrogating with a μ=50% tone modulated interrogation signal, wherein the x-axis and left y-axis data are normalized to the power that minimizes conversion loss

$$( {P}_{ht , f}^{↓} )$$

and CLₘᵢₙ, respectively;

FIG. 30A is a graph of measured channel fading versus track position, normalized to median of each measurement, for each of the forward link at frequency f (solid line) and the reverse link at frequency 2f and for the spatially-dependent fading example, wherein the harmonic transponder of FIG. 30C is interrogated using antenna A and the backscattered response is received using antenna B;

FIG. 30B is a graph of measured channel fading versus track position, normalized to median of each measurement, for each of the forward link at frequency f (solid line) and the reverse link at frequency 2f and for the spatially-dependent fading example, wherein the harmonic transponder of FIG. 30C is interrogated using antenna B and the backscattered response is received using antenna A;

FIG. 30C is a diagram illustrating the physical setup used in generating the graphs of FIGS. 30A and 30B;

FIG. 31 is a graph of cumulative probability versus relative loss of measured forward link (transmit antenna to harmonic transponder) fading at f, normalized to median, for the spatially-dependent fading example;

FIG. 32 is a graph of cumulative probability versus relative loss of measured reverse link (harmonic transponder to receive antenna) fading at 2f, normalized to median, for the spatially-dependent fading example;

FIG. 33 is a graph of fading, normalized to median, versus longitudinal position for outside chamber to inside chamber link measurements for the spatially-dependent fading example, wherein the scale is consistent with the scale of each of FIGS. 30A and 30B to show more benign, i.e., Rician, conditions;

FIG. 34 is a graph illustrating the spectrum of the transmitted amplitude modulated signal of a frequency-dependent fading example, showing the carrier-to-sideband ratio (C/SB_{tx,f}) to be ˜12 dB with the modulating tone of 10 kHz and μ=50% in the frequency-dependent fading example;

FIG. 35 is a combined graph comparing received carrier-to-sideband values (C/SB_{rx,2f}) (solid line) to single carrier conversion loss (CL) (dashed line) measured when interrogating with a μ=50% tone modulated interrogation signal for the frequency-dependent fading example, wherein the x-axis and right y-axis data are normalized to the power that minimizes conversion loss

$$( {P}_{ht , f}^{↓} )$$

and CLₘᵢₙ, respectively;

FIG. 36A is a graph of transmit power (Pₜₓ) required to reach the desired C/SB_{rx,2f} of 10 dB for both multipath (solid line) and anechoic (dashed line) environments for the frequency-dependent fading example;

FIG. 36B is a graph of power received (Pᵣₓ) required to reach the desired C/SB_{rx,2f} of 10 dB for both multipath (solid line) and anechoic (dashed line) environments for the frequency-dependent fading example;

FIG. 37A is a graph of forward link fading versus frequency, i.e., the forward channel frequency response, for the frequency-dependent fading example;

FIG. 37B is a graph of reverse link fading versus frequency, i.e., the reverse channel frequency response, for the frequency-dependent fading example;

FIG. 38 is a graph of cumulative probability versus relative amplitude, showing the frequency selective fading statistics for the forward and reverse wireless links for the frequency-dependent fading example; and

FIG. 39 is a graph showing the bidirectional channel response, including nonlinear effects of the harmonic transponder, for the frequency-dependent fading example.

### DETAILED DESCRIPTION

At a high level, aspects of the present disclosure include methods for interrogating a nonlinear transponder to determine its operating point once it has been fully assembled and/or deployed in the field. This disclosure includes empirical data demonstrating the ratios between the intermodulation distortion products produced when interrogating with two closely spaced frequencies will change in a well-behaved manner with changes in the operating point of the transponder. The present disclosure also includes empirical data demonstrating that the amplitude modulation (AM) characteristics of a backscattered signal resulting from a tone-modulated AM interrogation signal will change in a well-behaved manner with changes in an operating point of the transponder. Several uses of such methods are also presented herein.

Aspects of the present disclosure include over-the-air, i.e., wireless, methods to discern an operating point of a nonlinear transponder and subsequently determining bidirectional losses between the transponder and an interrogator. In some examples, the over-the-air methods leverage intermodulation distortion products (IMDs) to ascertain the transponder operating point. In some examples, the methods may also or alternatively include utilizing an interrogation signal that is an AM carrier wave modulated using a relatively lower-frequency signal, such as a constant-tone signal, and comparing AM characteristics of components, such as carrier-to-sideband characteristics, of the backscattered signal to those of the interrogation signal. As described below in connection with examples, over-the-air interrogations methods of the present disclosure can be used for a variety of purposes such as, but not limited to, analyzing one or more environmental conditions, for example, determining one or more physical properties of media, such as concrete, snowpacks, soils, and geotechnic structures, such as railroad ballast, roadbeds, rip-rap-type slope stabilizers, etc., characterizing multipath in wireless communications channels, antenna mapping, and quality control, among others. Relatedly, various systems for enabling the foregoing and other purposes and well as software for performing over-the-air methods and application-specific methods particular to applications of the over-the-air methods disclosed herein are also described below.

### I. EXAMPLE METHODS OF INTERRROGATING NONLINEAR TRANSPONDERS

### I.A Harmonic Transponders

For the sake of illustration, features, aspects, and functionalities of the present disclosure are described herein in the context of second-order harmonic transponders, which are a common type of nonlinear, and frequency-multiplying, transponders. While the examples focus on second-order harmonic transponders, those skilled in the art will readily appreciate that other nonlinear transponders can be used, such as harmonic transponders that output, or backscatter, a signal containing one or more higher-order harmonics of the interrogating signal or backscatter a signal containing one or more other multiples of the interrogating signal. In addition, those skilled in the art will readily understand from the examples provided herein how to implement the broad principles disclosed herein using only ordinary skill in the art and without undue experimentation.

Harmonic transponders are passive devices that have been demonstrated for a variety of tracking and sensing applications, including tracking insects, locating buried infrastructure, and microfluidics sensing. A significant advantage of a harmonic transponder compared to a mono-frequency transponder, such as a radio-frequency identification tag (RFID), is that backscatter and ground-clutter interference are effectively eliminated, since the interrogator receiver is tuned to receive only the harmonic tone of interest. Because of this advantage, a harmonic transponder can have an activation level nearly 20 dB below that of RFIDs (i.e., <−30 dBm), and therefore is able to be interrogated at greater distances, at lower power, and/or while embedded in a medium that can significantly attenuate the interrogation signal (e.g., soil).

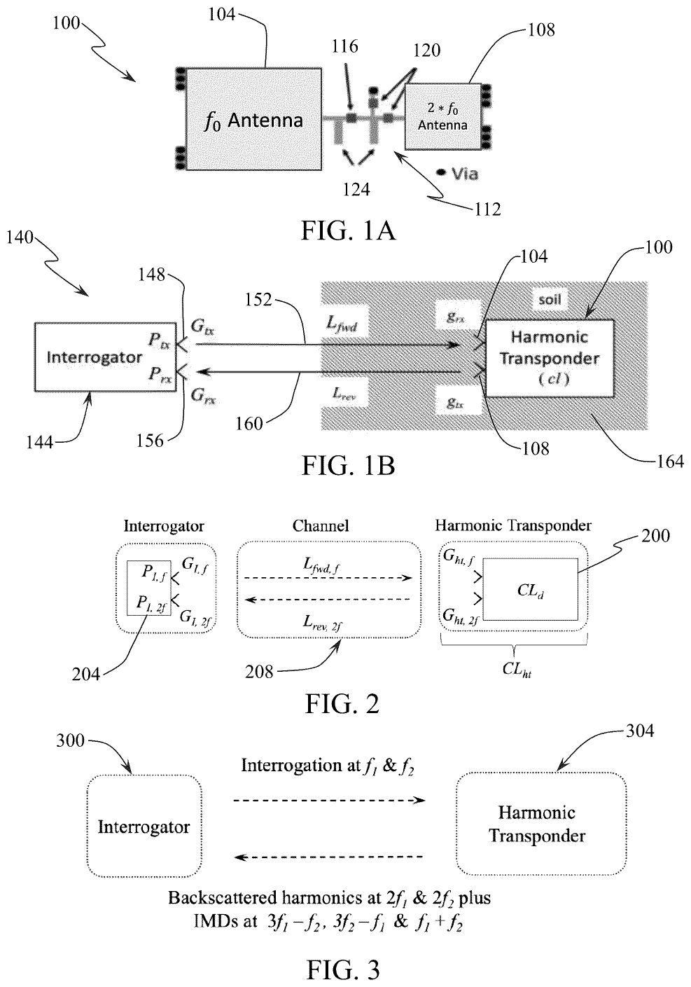

FIG. 1A illustrates an example second-harmonic (2H) transponder 100 that can be used with methodologies, devices, and systems disclosed herein. In this example, the 2H transponder 100 includes a receiving antenna 104, a transmitting antenna 108, and circuitry 112 electrically connecting together the receiving and transmitting antennas. In this example, the circuitry 112 includes a diode 116, inductors 120, and tuning stubs 124 that, among other things, double the frequency, f₀, of an interrogating signal (not illustrated) that the 2H transponder 100 receives at the receiving antenna 104 to create the doubled frequency (2*f₀, or simply 2f₀) of the backscattered signal (not illustrated) output by the transmitting antenna 108. Those skilled in the art will readily understand how to design working embodiments of the illustrated 2H transponder 100 and similar transponders, such that further explanation is not required to practice the inventions of the appended claims without undue experimentation.

FIG. 1B illustrates a sensor system 140 that includes the 2H transponder 100 of FIG. 1A, as well as an interrogator 144 that transmits an interrogation signal (not illustrated) via a transmitting antenna 148 over a forward over-the-air link 152 and receives, via a receiving antenna 156, a backscattered signal (not illustrated) over a reverse over-the-air link 160. With reference to FIG. 1B, the power of the received backscattered signal (Pᵣₓ at 2×f₀), is dependent on the effective transmitted power (Pₜₓ×Gₜₓ at f₀), the forward link loss (L_{fwd}) on the forward link 152, the conversion loss (gᵣₓ×cl×gₜₓ of the 2H transponder 100 that consists of the gain (gᵣₓ) of the receiving antenna 104, conversion loss of the diode 116 (FIG. 1A), and the gain of the transmitting antenna 108, respectively), the reverse link loss (Lᵣₑᵥ) on the reverse link 160, and the gain (Gᵣₓ) of the receiving antenna 156 of the interrogator 140, as shown (in log form) in the following Equation 1.

$${P}_{rx} = {\underbrace{{P}_{tx} + {G}_{tx}}}_{EIRP} - {L}_{fwd} + {\underbrace{{g}_{rx} - cl + {g}_{tx}}}_{harmonic transponder} - {L}_{rev} + {G}_{rx} \qquad ( 1 )$$

wherein “EIRP” denotes the effective isotropic radiated power.

The 2H transponder 100 is a nonlinear device, because the conversion loss (cl) of the diode 116 has a nonlinear dependency on the incident power at the 2H transponder. This incident power is dependent not only on Pₜₓ, Gₜₓ and L_{fwd}, but also on the effective gain of the receiving antenna 104 of the 2H transponder 100 in the direction of the interrogation signal (gᵣₓ). Therefore the reradiated signal at, e.g., 2×f₀ has a nonlinear dependency on changes in forward link loss (L_{fwd}) and/or the orientation of the 2H transponder 100 (impacting gᵣₓ). In addition, the received backscatter power depends linearly on the transponder's transmit antenna gain/orientation (gₜₓ) and losses in the reverse link 160 (Lᵣₑᵥ).

For a given measurement, there are typically certain knowns that appear in Equation 1. Particularly, the performance of the interrogator 140, particularly its effective isotropic radiated power (EIRP=Pₜₓ×Gₜₓ) and Gᵣₓ. Still, for an over-the-air measurement, the operating point (OP) for the device, which is defined herein as the power incident at the diode 116 (FIG. 1A) of the 2H transponder 100, will depend on unknowns, as shown (also in log form) in the following Equation 2.

$$OP = EIRP {\underbrace{- {L}_{fwd}}}_{unknown ?} + {\underbrace{{g}_{rx}}}_{uncertain ?} \qquad ( 2 )$$

Under free-space/idealized channel conditions, one can calculate the forward and reverse link losses and determine the OP if some knowledge of the orientation of the 2H transponder 100, and therefore gᵣₓ, is assumed. However, when the device is embedded in a medium 164 with unknown loss (e.g., soil, concrete, snowpack, etc.), then, using existing methods, the forward link loss cannot be isolated from the conversion loss and from the reverse link loss. This is particularly true when the medium 164 has different loss characteristics at f₀ (forward link) and 2×f₀ (reverse link, in this example), due to, for example, frequency-dependent dielectric properties.

The ability to isolate these link losses has a variety of applications. By way of example and not limitation, there are applications in agriculture, infrastructure monitoring, and environmental investigation and monitoring, among others. As a nonlimiting example, a nonlinear transponder may be buried in soil, such as the 2H transponder 100 of FIG. 1B embedded in the medium 164 (soil in this example), to monitor changes in moisture, which will change the strength of the received backscatter signal. As described more herein, by determining the incident power at the embedded transponder, then the forward link loss and the conversion loss of the transponder can be discerned. The remaining bidirectional link loss can be attributed to the reverse link. Knowing the link losses due to soil attenuation for different frequencies will allow for finding the moisture content for the soil layer between the air boundary and the embedded transponder (vs. a point measurement, which a typical sensor might make). Additional information on the correlation between link loss and soil moisture can be found in J. Frolik, J. Lens, M. Dewoolkar, and T. Weller, “Effects Of Soil Characteristics On Passive Wireless Sensor Interrogation,” IEEE Sensors Journal, vol. 18, no. 8, April 2018, which is incorporated by reference herein in its entirety. These general principles can be extended to a variety of media types other than soil. The present disclosure includes systems and methods for isolating the OP of the embedded transponder, such as the 2H transponder 100 of FIG. 1B.

### I.B Multi-Frequency Interrogation

Aspects of the present disclosure include a new approach for interrogating a nonlinear transponder, with the objective of determining the power at the input to a diode of the transponder, such as, for example, at the diode 116 of the 2H transponder 100 of FIG. 1A. This power determines the nonlinear OP of the transponder, and thus the relative power ratios of harmonics and IMDs that the transponder will produce.

As noted above, the OP of a harmonic transponder may be characterized by analyzing backscattered signals, for example, in a band about the second harmonic of an interrogation signal. Specifically, the interrogator transmits two closely-spaced frequencies (f₁ and f₂) and measures the returns at (1) the second harmonics (2×f₁ and 2×f₂), (2) the second-order IMD (f₁+f₂), and (3) the fourth-order IMD (3×f₁−f₂ and 3×f₂−f₁). In an example the two signals at the two frequencies are transmitted at substantially the same time, the simultaneous transmission of the two frequencies resulting in the IMD products. The first and second frequencies may be any frequency in the radio frequency spectrum. In an example the interrogator may be configured to filter out or otherwise eliminate any IMD products between the two frequencies of the interrogation signal from the forward link, for example eliminate any IMDs upstream of the transmit antenna of the interrogator.

To measure the backscattered signal the interrogator may include a spectrum analyzer configured to a nominal center frequency at a midpoint between 2×f₁ and 2×f₂ and configured to sweep across a bandwidth where the IM4 and 2H components are expected. The interrogator (or other device used to measure the backscattered signal may include a power detector circuit and a power measurement module configured to process the measured backscattered signal to determine a level at specific frequencies. In one example one or more of the functions performed by the interrogator, including generating the interrogation signals and measuring or characterizing the received backscattered signal can be performed with a appropriately configured software defined radio.

As will be appreciated by persons having ordinary skill in the art, the disclosure is not limited to specific harmonics and IMDs and can be applied to harmonics other than second harmonics and IMDs other than the ones utilized in the example implementations disclosed herein. In an example the two closely-spaced frequencies (f₁ and f₂) are selected according to a nominal bandwidth and peak response frequency of the harmonic transponder. The two interrogation frequencies are selected such that the second harmonics and the IM4 products of the selected two frequencies are within the response bandwidth of the transponder.

The relative power ratios between these three distortion components, as measured by the interrogator (or other device functioning as an interrogator), provide a unique signature for the OP of the transponder. That is, these ratios depend only on the power incident to the diode of the transponder (due to interrogator's effective transmit power, forward link loss, device's receive antenna) and the diode characteristics versus the OP, which can be determined in advance, and not on the return link (which is composed of the transmitting antenna of the transponder, reverse link loss, and the receiving antenna of the interrogator).

With reference to FIG. 2, following is a slightly modified approach relative to Equations 1 and 2, above, to determining a unique signature for an operating point of a nonlinear transponder, here, a 2H transponder 200, via an interrogator 204 via an over-the-air channel 208. In this example, the interrogator 204 sends a signal of known frequency (f) and power and measures the power of the backscattered harmonic (2f). In this example, the conversion loss used is the conversion loss of the 2H transponder 200, referred to as “CLₕₜ”. The power of the received backscattered signal (P_{I,2f}), is dependent on the EIRP (P_{I,f}×G_{I,f}) of the interrogator 204, the forward link loss (L_{fwd,f}), the conversion loss (CLₕₜ=G_{ht,f}×CL_{d}×G_{ht,2f}) of the 2H transponder 200, the reverse link loss (L_{rev,2f}), and the receiving antenna gain (G_{I,2f}) of the interrogator, as shown (in log form) in the following Equation 3.

$${P}_{1 , 2 f} = EIRP = {L}_{fwd , f} - {\underbrace{{CL}_{ht}}}_{nonlinear} - {L}_{ref , 2 f} + {G}_{I , 2 f} \qquad ( 3 )$$

Note that as the conversion loss (CLₕₜ) of the 2H transponder 200 is nonlinear, as the conversion loss (CL_{d}) of its diode has nonlinear dependency on its incident power. Thus the reradiated signal at 2f has a nonlinear dependency on changes in forward link, i.e., EIRP, loss (L_{fwd}) and/or the orientation of the 2H transponder 200 (impacting G_{ht,f}). At the same time, the received backscatter power depends linearly on the harmonic transponder's transmit antenna gain/orientation (G_{ht,2f}) and the reverse link (i.e., G_{I,2f}, L_{rev,2f}).

For a given measurement, there should be certain knowns that appear in Equation 3. Particularly, the performance of the interrogator should be known, i.e., its EIRP and G_{I,2f}. For an over-the-air measurement, the OP for the 2H transponder 200, defined herein as the power incident at the 2H transponder when normally oriented, will depend on unknowns, as shown (also in log form) in the following Equation 4.

$$OP = EIRP {\underbrace{- {L}_{few , f}}}_{known ?} + {\underbrace{{G}_{ht , f}}}_{orientation certain ?} \qquad ( 4 )$$

Referring now to FIG. 3, this figure illustrates an example using an interrogator 300 to transmit an over-the-air interrogation signal containing two interrogating frequencies, f₁ and f₂. FIG. 3 also shows the corresponding components of the over-the-air return signal backscattered by a 2H transponder 304 that fall in a band around the second harmonic of the interrogation signal and that can be used to determine the OP of the 2H transponder. Specifically, in this nonlimiting example the interrogator 300 transmits two relatively closely spaced frequencies f₁ and f₂ and measures the returns at (1) the second harmonics (2f₁ and 2f₂; “2H”), (2) the second-order IMD products (f₁+f₂; “IM2”), and (3) the fourth-order IMD products (3f₁−f₂ and 3f₂−f₁; “IM4”).

As shown below, the relative power ratios between these three distortion components, as measured by the interrogator 300, provide a unique signature for the OP of the 2H transponder 304. That is, these ratios depend only on 1) the power incident at the 2H transponder 304 when normally oriented (due to interrogator's effective transmit power and forward link loss) and 2) the characteristics of the diode of the 2H transponder vs. OP (known), and not at all on the return link, which is composed of 2H transponder's transmitting antenna (not shown), the reverse link loss, and the receiving antenna (not shown) of the interrogator 300.

### I.C Results From Example Implementation

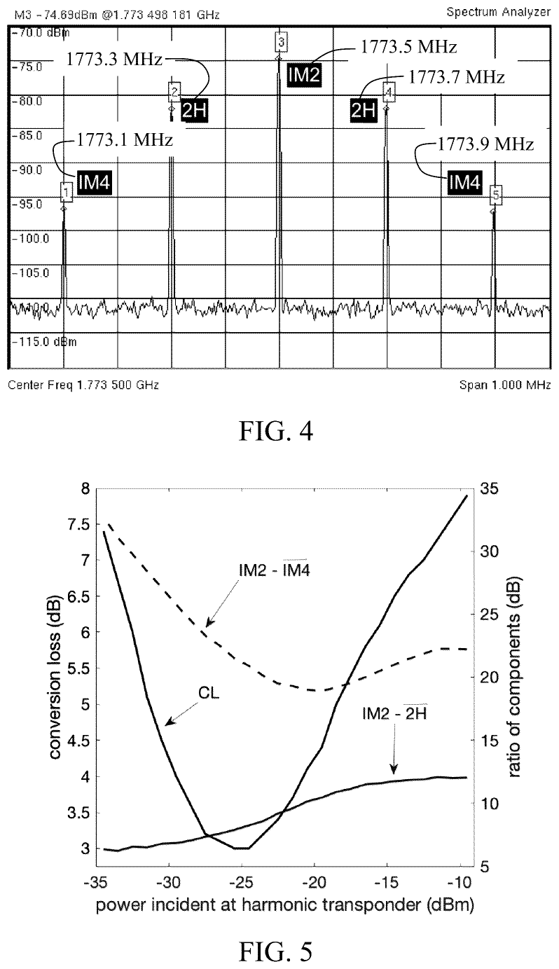

To illustrate the general multi-frequency approach, a commercial 2H transponder (available from RECCO AB, Radiovägen, Sweden; not shown but similar to the 2H transponders 100, 200, and 304 of, respectively, FIGS. 1A, 2 and 3) for the purpose of locating avalanche victims was used. The 2H transponder was placed in an anechoic environment, and an interrogation signal was sent to the transponder from a distance of 1 m and with an EIRP of ˜10 dBm, using two frequencies near 886.75 MHz (f₁=886.65 MHz and f₂=886.85 MHz). Using a spectrum analyzer (e.g., resident with an interrogator, such as any of the interrogators 144, 204, and 300 of, respectively FIGS. 1B, 2, and 3), the return was measured in the 1.77 GHz band, the results being shown in FIG. 4, and consisted of the second harmonics of the interrogation signals and their second-and fourth-order IMDs (referred to herein and in the appended claims as, respectively “IM2” and “IM4”). From left to right in FIG. 4 is the lower IM4 product (3f₁−f₂=1773.1 MHz), the second harmonic (2H) of f₁ (2f₁=1773.3 MHz), the IM2 product (f1+f₂=1773.5 MHz), the second harmonic of f₂ (2f₂=1773.7 MHz), the upper IM4 product (3f₂−f₁=1773.9 MHz). As the transmit power of the interrogating signal was decreased, the ratios between these harmonics and IMDs changed due to the nonlinear nature of the 2H transponder.

The transmitted EIRP was then adjusted in 1 dB increments/decrements over a 25 dB range, and at each step two measurements were made. The first was a single carrier-frequency measurement at f=886.75 MHz, with the second being the backscattered response signal measured at 2f. Equation 3, above, was solved for the conversion loss, CLₕₜ, of the 2H transponder, and these data are plotted using the lefthand y-axis of FIG. 5 (conversion loss).

The second measurement leverages the dual-frequency approach described above. Using, for example, the above-mentioned spectrum analyzer, the power of the five distinct frequencies seen in FIG. 4 were measured. Note that there are two 2H components at 1773.3 MHz and 1775.7 MHz and two IM4 components at 1773.1 MHz and 1773.9 MHz. As such, this example considers the single IM2 component (f₁+f₂) at 1773.5 MHz as a reference for calculating the ratios between these distortion products. Specifically, for example, it is here desired to quantify a ratio between the IM2 component and the mean of the two second harmonic components ( [symbol] ), and a ratio between the IM2 component and the mean of the two IM4 components (IM̅4). For an OP dynamic range of 25 dB, these ratios (differences in dB) for (IM2- [symbol] ) and (IM2-IM̅4) are plotted in FIG. 5 (the righthand y-axis provides the scale).

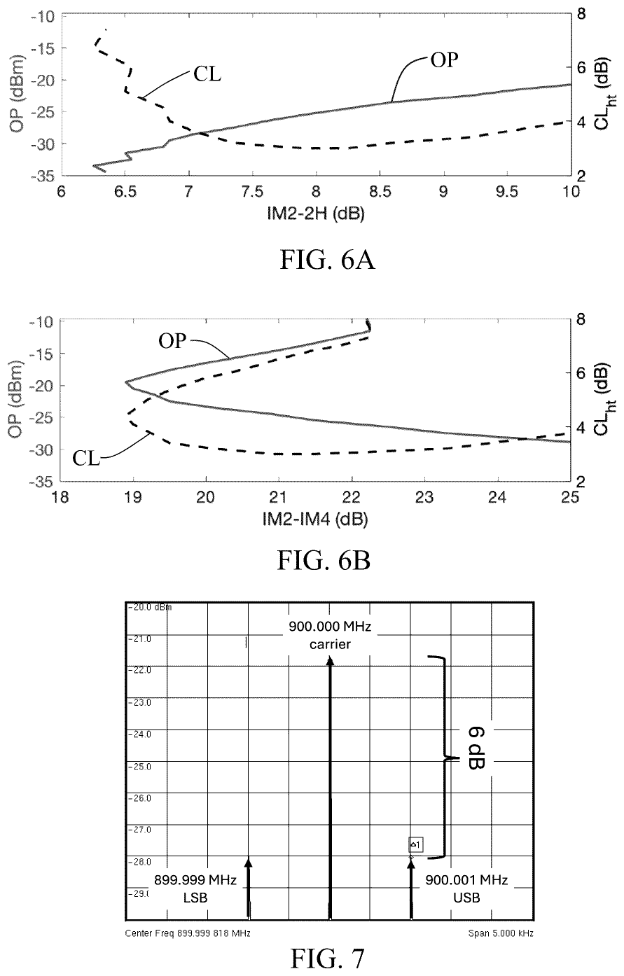

With the two well-behaved curves of FIG. 5, an inverse problem containing measured IMD product ratios can be readily solved to determine the OP of the device. FIGS. 6A and 6B show this inverse mapping, wherein the measurable ratios can provide the OP of the 2H transponder (lefthand y-axis) and the conversion loss CLₕₜ (righthand y-axis).

As an example, consider the measured data shown in FIG. 4, from which the following ratios can be found: IM2- [symbol] =7.6 dB and IM2-IM̅4=22.8 dB. Using FIG. 6A, the IM2- [symbol] ratio corresponds to an interpolated OP of −26.5 dBm, while using FIG. 6B, the measured IM2-IM̅4 ratio results in an interpolated OP of −26.9 dBm. Both results are within 0.5 dB of the actual −27 dBm incident power. These results indicate that over a 25 dB range, and using an over-the-air approach, the incident power at a remote nonlinear transponder can be found with good accuracy using only the signals measured at an interrogator.

### I.D Constant-Tone Amplitude Modulation

In some examples, the OP of a nonlinear transponder can be uniquely identified in unknown channel conditions by leveraging an over-the-air AM interrogation signal that is amplitude modulated, for example, using a low-frequency tone. The channel loss can be determined by comparing the AM characteristic components, such as sideband frequencies, of the backscattered signal returned from the nonlinear transponder to similar components of the interrogation signal. Tone amplitude modulation is provided below by way of example. However, other modulation techniques may also or alternatively be utilized. For example, a modulation waveform other than a sinusoid, such as a square wave, may alternatively be used. In some examples a single AM sideband rather than two AM sidebands may be used.

Typically a harmonic transponder is interrogated with a sinusoid having fixed frequency, f=f_{c}. In examples disclosed herein, the interrogation signal sinusoid is a carrier signal, f_{c}, amplitude modulated with a lower frequency, fₘ, tone. In tone AM, a low frequency baseband tone, m(t)=M·cos(2πfₘt), modulates a high frequency carrier, c(t)=A·cos(2πf_{c}t), resulting in the signal seen in Equation 5, below. The carrier frequency, f_{c}, is the interrogation frequency at which the harmonic transponder is designed to operate (e.g., in microwave range), while fₘ is typically on the order of kHz. The resulting double-sideband, large carrier (i.e., AM) signal is given by the following Equation 5.

$${ϕ}_{AM} ( t ) = A * [ 1 + μ \cdot cos ( 2 π {f}_{m} t ) ] * cos ( 2 π {f}_{c} t ) \qquad ( 5 )$$

wherein μ=M/A is the modulation index, with M being the magnitude of the tone and A being the magnitude of the carrier.

The modulation index, μ, describes the relative depth of m(t) with respect to the carrier in the time domain. μ manifests itself in the frequency domain as a carrier-to-sideband ratio (C/SB) between the power in the carrier frequency, at f_{c}, and the two sidebands, at f_{c}−fₘ and f_{c}+fₘ. The C/SB ratio is minimized for μ=100%, where each sideband has one quarter the power of the carrier, giving a C/SB of 6 dB. As μ decreases, C/SB increases. For u values of 75%, 50%, and 25%, the C/SBs are theoretically 9 dB, 12 dB and 18 dB, respectively. In a linear channel, this C/SB ratio will be preserved at the receiver. However, with a nonlinearity introduced by the harmonic transponder, it was found that the C/SB is not always preserved.

The efficiency, η, of amplitude modulation is given by the power in the sideband signals, P_{S}, to the total power, which is the power in the sidebands added to the power in the carrier signal, P_{C}=A²/2. That is, efficiency η=P_{S}/(P_{C}+P_{S}). For tone modulation, the power in the sideband signal is that of the lower sideband (LSB) added to the power in the upper side band (USB) (see, e.g., FIG. 7).

For tone modulation with a certain modulation index, μ, the AM efficiency is given by η=μ²/(2+μ²) and is maximized when μ=1 (i.e., 100%), giving η=⅓, and from the foregoing the total sideband power is P_{S}=(μA)²/4. Spectrally, for this case, the total power in two sidebands is one half that of the carrier, and therefore each sideband's value is one fourth that of the carrier, giving a C/SB of 6 dB (FIG. 7). For other modulation indexes, μ can similarly be mapped to η, which can be mapped to measurable C/SB. Likewise, measurable C/SB can be mapped to η and then to μ. For modulation indexes less than 100%, the C/SB ratios will increase in a manner driven by Equation 5, above. For linear channels, the C/SB ratio at the receiver will be the same as the C/SB ratio at the transmitter. However, for interrogation of a harmonic transponder, the bidirectional channel is nonlinear.

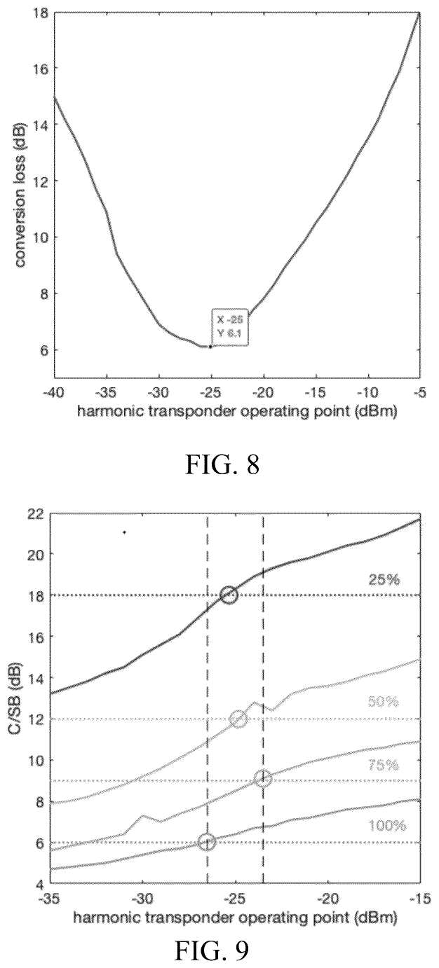

To illustrate the impact of a nonlinear channel on C/SB, η, and μ, a 2H transponder having the configuration of the 2H transponder 100 of FIG. 1A was tested over a 2 m link in an anechoic environment (not shown). The 2H transponder was interrogated with a signal having carrier frequency f_{c}=886.75 MHz, which was modulated with a tone having fₘ=10 kHz. With modulation off, the conversion loss versus operating point of the 2H transponder was characterized, as shown in FIG. 8.

As shown in FIG. 9, if one compares the C/SB received at the interrogator (y-axis) to the known C/SB transmitted by the interrogator (shown by the horizontal lines), then one can remotely determine the OP of the 2H transponder within ±1.5 dB. In addition or alternatively, the modulation indices of the transmitted and received signals can be compared to determine the OP. For example, a computer-implemented method of interrogating a nonlinear transponder may include storing in memory of an interrogator or other device a predetermined transponder signal-to-OP relationship, such as the relationships shown in FIG. 9. Any implementation may be used, such as a dataset, lookup table, equation, etc., that correlates measured characteristics such as the C/SB ratios shown on the y-axis of FIG. 9, or modulation index ratios, to the operating point of the transponder. Such a method may also include machine-executable instructions stored in memory that cause a processor to perform operations that include determining an operating point of the transponder according to measured characteristics of the transponder signal and the transponder signal-to-OP relationship. In one example, one or more of the functions performed by an interrogator, including generating the interrogation signals and measuring or characterizing the received backscattered signal can be performed with, for example, an appropriately configured software defined radio.

The implications of this result for characterizing an unknown channel loss are not insignificant. Consider again the nonlimiting example scenario where a harmonic transponder is embedded in soil and interrogated from above ground with the objective of measuring soil moisture. As soil retains and loses moisture, the forward and reverse channel losses will increase and decrease, respectively, and in differing amounts due to the different frequencies (f and 2f, respectively). Methodologies disclosed herein allow the OP of a nonlinear transponder to be ascertained and from which the forward channel loss, L_{fwd}, can be found using Equations 1 and 2, above.

Once the OP of the nonlinear transponder is found, the transponder's conversion loss, CL, can also be found using calibration data, such as the data illustrated in FIG. 8. With L_{fwd} and CL found, Equation 1, above, rewritten as follows can be used to solve for the reverse channel loss, Lᵣₑᵥ.

$${L}_{rev} = {\underbrace{EIRP + {G}_{rx , 2 f}}}_{fixed @ interrogator} {\underbrace{- {P}_{rx}}}_{measured} {\underbrace{- {L}_{fwd}}}_{Eq . ( 2 )} {\underbrace{- CL}}_{FIG . 8} - Opt \qquad ( 6 )$$

With L_{fwd} known at f and Lᵣₑᵥ known at 2f, the parameter to be sensed, e.g., soil moisture, for the medium embedding the harmonic transponder, e.g., soil, can be found from known dielectric properties. In a computer implemented method the calibration data may be stored in memory in any form known in the art, such as a dataset, lookup table or equation that correlates CL to OP. The method may also include instructions stored in memory that cause a processor to perform operations that include determining the CL of the transponder according to the determined OP and the CL−OP relationship.

As seen in FIG. 8, CL is minimized for this the subject harmonic transponder at −25 dBm. As seen from FIG. 9 that at this OP, the C/SB received is nearly equivalent to that of the C/SB transmitted. That is, the shape of the envelope of the backscattered signal remains the same, even though the carrier frequency has doubled. For higher operating points, it is found that the C/SB of the backscattered signal increases, whereas the opposite is true as the operating point decreases. By conducting multiple, independent interrogations with different values of μ, more accurate values of a transponder's OP can be found and thus more accurate characterizations of channel loss.

### I.D.1 Using the AM Approach to Remove Uncertainty in Forward and Reverse Propagation Loss Measurements

As noted above in section I.B, an important metric in characterizing the performance of a harmonic transponder is its CL, which is the ratio (in dB) of the power of the microwave signal incident at the device (at a single frequency “f”) to the power of the reradiated microwave signal (at frequency “2f”). The CL of a harmonic transponder can be found by measuring the reradiated signal as the power of the incident signal is changed. FIG. 8 illustrates the resulting conversion loss curve as discussed above. In the plot of FIG. 8, the minimum conversion loss is found to be 6.1 dB, when the incident power (i.e., operating point, OP) is −25 dBm. These data were collected in an anechoic testing chamber, which is ideal for over-the-air testing. Under non-ideal conditions, measurement variations of +/−0.5 dB are not uncommon. As a result, determining the minimum CL and the associated OP are prone to significant errors.

The difficulty of collecting CL data was demonstrated when the channel between the interrogator and transponder was a snowpack. A 0.5 m snowpack was constructed over a ground-based harmonic transponder, using sifted late season snow (45% moisture content). An interrogation system was located 1.4 m away from the device. A continuous-wave (CW), single-frequency, 1.2 GHz wireless signal was transmitted to the device and its power was swept across a 15 dB range (EIRP of ˜25 to 40 dBm) at 1 dB increments. A spectrum analyzer was used to analyze the received signal at 2.4 GHz.

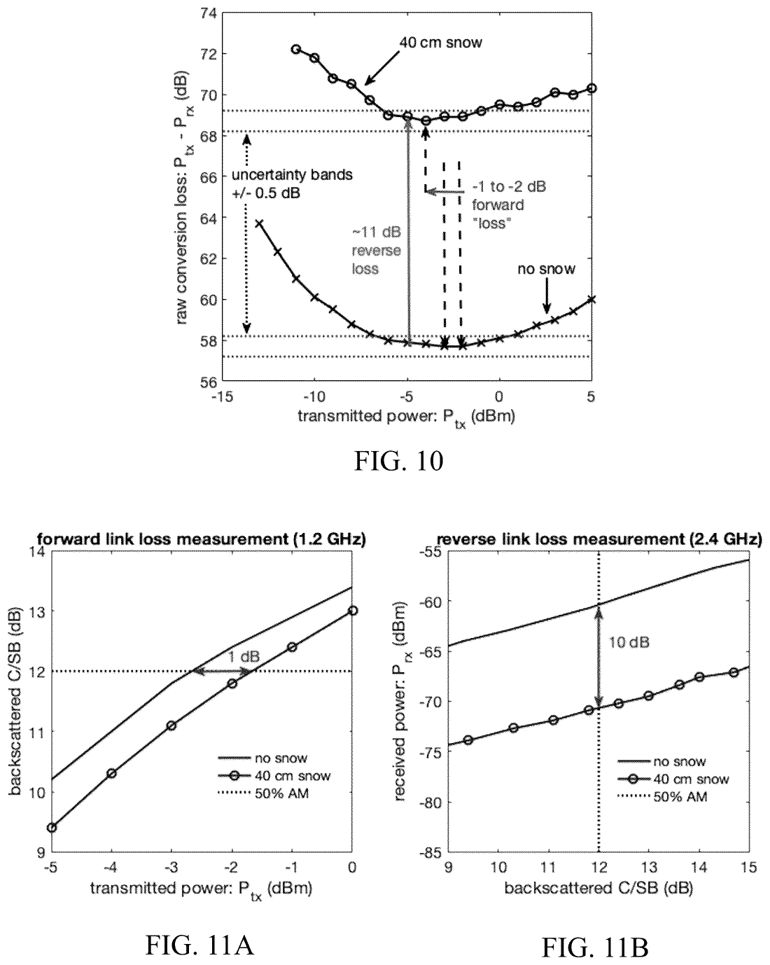

The power of the backscattered signal was measured at each increment of transmit power, and the difference between the transmitted and received signals were plotted against the transmitted power, with the plot shown in FIG. 10. In these data of FIG. 10, “raw” conversion loss is shown, which is based on measured values prior to removing any fixed calibration offsets. An example measurement at 40 cm depth is presented, along with the case of having no snow. The vertical dashed arrows in FIG. 10 show the minimum points of each of the curves. In the “no snow” case, the same raw CL value was measured at two different powers. The horizontal dotted lines indicate +/−0.5 dB from the minimum point in each curve, and can be considered a nominal uncertainty region for the measurement. It is notable that both curves remain within this uncertainty region for several dB (i.e., >+/−2.5 dB in transmitted power in both cases).

Using these data, one can attempt to identify the losses due to the forward link and the reverse links. Any shift in OP (horizontal axis) would be attributed to forward link losses. Using these data, this shift is −1 dB to −2 dB, meaning the snow increased the power received by the transponder by that amount, which is not a physical possibility. The vertical shift is attributed to reverse link loss, but also includes any changes in the transponder's diode loss. Here it can be inferred that the loss to be ˜11 dB. In both loss measurements, there is uncertainty in which data to use to ascertain the losses, resulting in values that are inexact.

In contrast to using a single CW signal that is needed to ascertain CL, the AM interrogation approach uses a modulated carrier, which has three signal components: one carrier and two sidebands. FIGS. 11A and 11B show data collected for the same snowbank scenario but when the AM approach with a 50% modulation index was used. Under this scenario, the carrier to sideband ratio of the sent signal is 12 dB. When the C/SB ratio of the measurable backscattered signal is also 12 dB, that indicates the device is at its most linear operating point. This fact was used to define the points at which to make our forward and reverse link loss measurements.

FIG. 11A shows the shows that approximately 1 dB more power needs to be transmitted to achieve the C/SB=12 dB operating point when the harmonic transponder is buried under 40 cm of snow. That is, the snowpack attenuates the 1.2 GHz signal 1 dB. FIG. 11B shows the shows that when the harmonic transponder is at the 12 dB C/SB operating point, the power received when under 40 cm of snow is 10 dB less than when the snow is not present. Under both conditions, the device is at the same operating point, so this 10 dB difference can be attributed to only the change in loss in the reverse link. These results demonstrate that using the AM approach removes uncertainty associated with what data to use to make the link loss measurements.

### II. Example Applications

As noted, the methods disclosed herein allow one to isolate losses related to the forward link (L_{fwd}), as seen in FIG. 1B and Equation 1, above, from the remainder of the bidirectional link. This section provides several example applications for such a method. Those skilled in the art will readily appreciate that the following examples are merely illustrative and that methods disclosed herein may be utilized in any application involving interrogation of nonlinear transponders using the fundamental principles disclosed herein and variations thereof and derivable therefrom that would be apparent to those skilled in the art after reading the present disclosure.

### II.A Soil Moisture Measurements

### II.A.1 Dual-Frequency Interrogation

Propagation loss through a layer of soil depends on the soil type, depth of the soil layer, and soil moisture, along with the frequency of the signal. In an example application, harmonic transponders buried in soil are utilized for long-term and low-cost soil moisture monitoring. In this application, the attenuation seen on the forward link will differ (and will typically be less) than the attenuation seen on reverse link, particularly as soil moisture increases, due to the difference in signal frequencies.

In one example an interrogator sends a dual space-apart frequency (f₁ & f₂) interrogation signal and measure not only the returned power, but also the ratios between the returned components. These ratios will provide the OP of the buried transponder. Additionally or alternatively, the interrogator may transmit an AM signal and measure the amplitude modulation characteristics of the backscattered signal. The OP of the buried device may be additionally or alternatively determined from the measured amplitude modulation characteristics. An example using the AM interrogation approach is presented below in section II.A.2.

Knowing the OP provides the forward link loss and the transponder's CL, and subsequently the reverse link loss can be calculated. The forward and reverse link losses will consist of the loss through the layer of medium as well as the loss through the layer of air between the interrogator and the medium surface, such as the ground surface. The height of the interrogator above the ground can be utilized to determine the loss attributable to transmission through air so that the link loss through the layer of medium can be determined. In drone-based applications or other flight-based methods the drone may include one or more sensors known in the art for determining drone altitude for determining the air layer components of the forward and reverse link losses.

These losses, at both the forward and reverse link frequencies, can be used to determine the soil's moisture by utilizing data on loss vs. moisture as a function of frequency and depth, such as collected in: J. Frolik, J. Lens, M. Dewoolkar, and T. Weller, “Effects Of Soil Characteristics On Passive Wireless Sensor Interrogation,” IEEE Sensors Journal, vol. 18, no. 8, April 2018. Of particular note is that interrogation could be conducted regularly over an extended period time (e.g., years). This longitudinal time-series data may be used, for example, to establish baseline measurements for acceptable, too dry, and too wet conditions.

### II.A.2 Constant-Tone-Modulated AM Interrogation

### II.A.2.a Transponder Nonlinearity

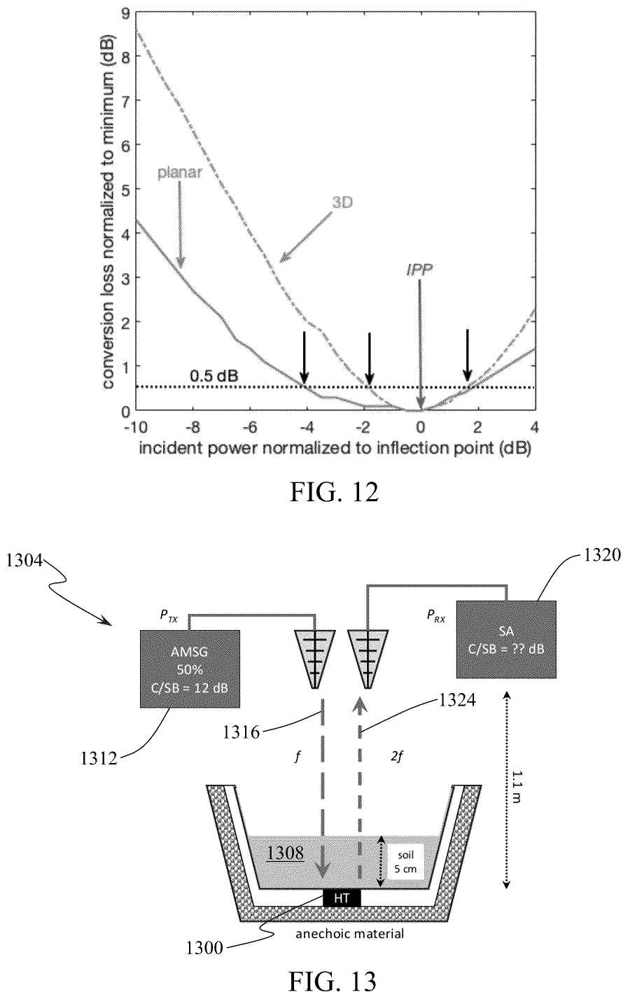

As noted above, the nonlinear behavior of a harmonic transponder is attributed to the presence of a diode. The impact of this nonlinearity is threefold. First, the nonlinearity creates frequency components at integer multiples of the incident frequency, which includes 2f, a backscattered return of interest. Second, the nonlinearity results in a device CL that is dependent on the incident power. This behavior is illustrate in FIG. 12 for two different harmonic transponders as described below. For both transponders, it is seen in FIG. 12 that CL is minimal at a specific incident power. In this disclosure, this point is referred to as the inflection power point (IPP) of the transponder. To collect these data, over-the-air absolute power measurements, which are prone to potential uncertainties of >+1 dB, were obtained. Considering that as little as +0.5 dB uncertainty in absolute power measurements, the error in determining the IPP can be significant. Thus, there is motivation to find a more accurate and precise means of remotely ascertaining the power incident at the relevant transponder.

Changes in CL relative to the input are a form of an AM-AM distortion effect, which is a common concern for power amplifiers in microwave systems. For link loss measuring purposes, the fact that this effect manifests itself in producing a backscattered C/SB that is nominally different than that of the interrogating signal.

### II.A.2.b Device Characterization

The AM-interrogation approach is demonstrated with two different harmonic transponder designs. One of the transponders was of a planar design that leveraged a silicon-based Schottky diode (HSMS-2820). This transponder operated with an interrogation frequency of 886.75 MHz. The other transponder had a compact 3D geometry and used a gallium arsenide (GaAs) Schottky diode (HSCH-9161). This transponder was designed to be interrogated at 1,185 MHz.

Referring to FIG. 13, the harmonic transponders (both denoted 1300) were interrogated using the setup 1304 seen in FIG. 13, but with no soil 1308 in place. In the setup 1304, an AM signal generator 1312, configured for μ=50% and C/SB=12 dB, was used to provide the interrogation signal 1316, and a spectrum analyzer 1320 was used to measure the C/SB of the backscattered signal 1324 as the transmitted power (P_{TX}) was varied. The AM carrier of the interrogation signal 1316 was set for the operating frequency of the corresponding transponder 1300 and amplitude modulated with a 10 kHz tone and with μ=25%, 50% or 75%. The transmitted power was varied about that which produced the IPPs in FIG. 12. In FIG. 12, the horizonal line at 0.5 dB and its associated arrows illustrate the potential range of error when measuring absolute power using over-the-air methods.

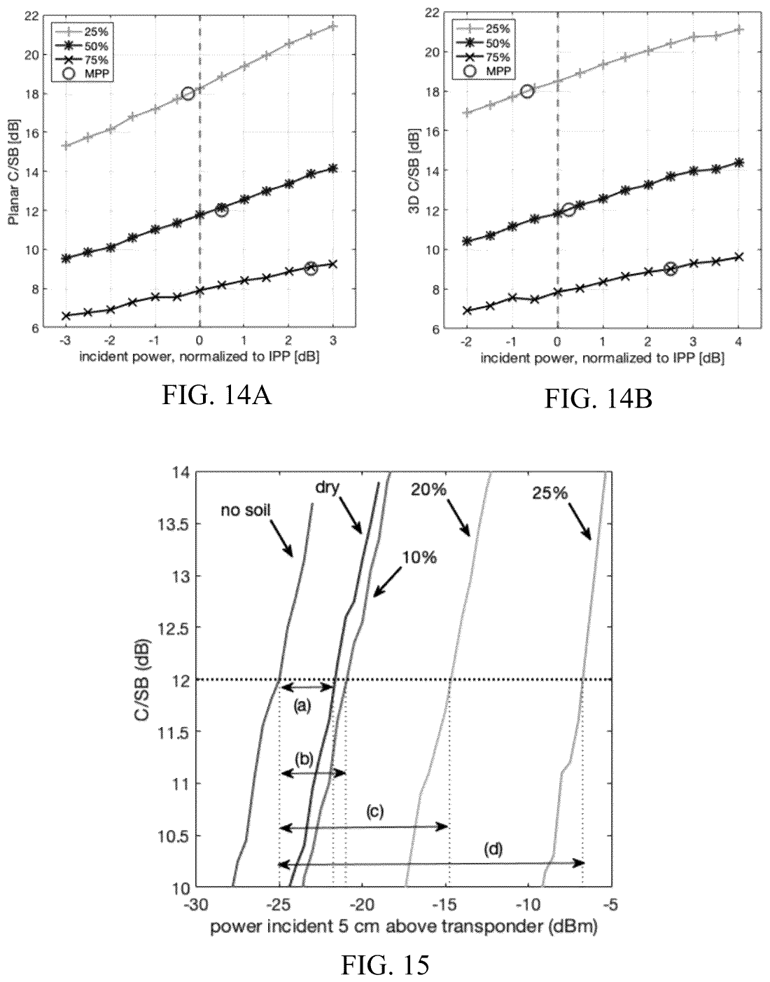

FIGS. 14A and 14B show the test results for the planar and 3D versions of the transponder 1300 shown in FIG. 13, respectively. Similar trends are seen for both versions of the transponder 1300. First, the AM-AM slope increases as μ decreases. Second, as the μ decreases, so does the signal's crest factor. It can be observed that results in the incident power at which the backscattered C/SB matches the interrogation C/SB becomes closer to the IPP. Recall that the IPP was determined from FIG. 12 when the carrier was not modulated, i.e., μ=0%. The incident power at this matched C/SB is referred to herein as the matched power point (MPP), which is noted on each of FIGS. 14A and 14B as a circle on each curve.

Because of the AM-AM slopes, and that C/SB is a ratio, the MPP can be found far more accurately and precisely than the IPP. These facts, as shown next, allow changes in path loss to be readily determined without relying on absolute power measurements, such as are needed to determine a transponder's CL. It is further noted that slopes associated with the planar design are steeper than the slopes associated with the 3D design, which is likely due to the different diode technologies used in each.

### II.A.2.c Measuring Soil Absorption

This example is related to monitoring the recovery of soils on slopes after severe wildfires. Such fires can significantly alter the soils to depths of several centimeters. But long-term soil moisture monitoring, along with knowledge of the nominal soil composition, can provide insight to the stability and strength of impacted slopes, and thus risks of landslides.

As a proof of concept that embedded harmonic transponders can determine absorption loss due to soil moisture, 5 cm of soil 1308 was placed above the planar harmonic transponder 1300, as illustrated in FIG. 13. The soil 1300 was mostly sand, with small amounts of silt and organics, representing preburn conditions (to serve as a baseline). Measurements were conducted at 0%, 10%, 20%, and, finally, at 25% gravimetric (i.e., by weight) water content, which is near the saturation point of the soil.

FIG. 15 show the μ=50% data collected for various soil moisture conditions to ascertain changes in the forward link loss. The trends exhibit expected behavior (i.e., more moisture results in greater loss). Each curve in FIG. 15 intercepts the 12 dB horizontal line at its MPP, with the changes in these MMPs corresponding to the changes in forward link loss. As those skilled in the art will readily appreciate, these forward link losses could be measured using any C/SB on this plot and/or at other μ. Aggregating such various data, it appears that resulting error in the link loss measurement can be <±0.25 dB.

### II.B Measuring Snow Water Equivalence

This example utilizes commercial off-the-shelf avalanche reflectors to ascertain snow water equivalences (SWEs) of snowpacks, such reflectors being a type of nonlinear, harmonic transponder. The particular transponders used are nominally interrogated with a signal of 900 MHz and then return (i.e., backscatter) a signal at twice this frequency (i.e., 1.8 GHz). It is noted that these transponders are also responsive when interrogated at higher frequencies, particularly at f=1.2 GHz, returning a measurable response at 2f=2.4 GHz. This latter frequency is known to be highly susceptible to the effects of water.

In this example, a 1.2 GHz carrier signal of the interrogation signal was modulated with a 10 kHz tone. The modulation process produced a spectrum with three components: the carrier at f_{c}=1.2 GHz and sidebands on both sides of f_{c} that are each 10 kHz away from f_{c}. Using a modulation index of 50%, the ratio between the sent carrier and these sidebands (C/SB ratio) is 12 dB. However and as described above, due to the transponder's nonlinearity, the C/SB ratio on the backscattered signal varies with a known dependency on the power incident at the transponder. As also described and illustrated above, by remotely measuring the backscattered power and the backscattered C/SB ratio, the propagation loss effects on backscattered signal can be isolated from any losses that impact the interrogation signal.

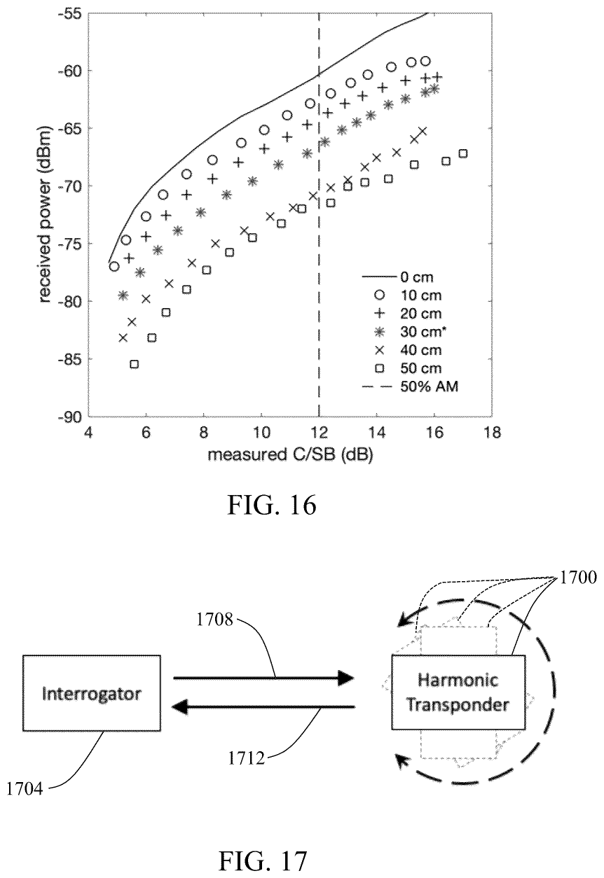

To illustrate the approach, a 0.5 m snowpack was constructed over a ground-based harmonic transponder using sifted late season snow (45% moisture content). An interrogation system was located 1.4 m away from the transponder. A spectrum analyzer was used to analyze the received signal. The 1.2 GHz signal's transmitted power was swept across a 15 dB range (EIRP of ˜25 to 40 dBm) at 1 dB increments. The backscattered signal's power, along with its C/SB ratio, was measured at each increment and then plotted against each other. These measurements were collected at 10 cm snowpack intervals. The results, shown in FIG. 16, show only the snowpack effects on the returned 2.4 GHz signal. These data indicate the nominal loss across the 0.5 m is ˜20 dB/m, corresponding to ˜45 dB/m SWE.

### II.C Receive Antenna Pattern Mapping

While the performance of harmonic transponder's receive and transmit antennas and diode can be separately characterized prior to the transponder's assembly, this is not the case after integration of these three components. Changes from these individual component measurements can occur due to, for example, impedance mismatch between either/both antennas and the diode, antenna pattern deformation caused by the object the transponder is deployed on (e.g., human body), and/or by the media the device is deployed in (e.g., soil, snow, etc.). As such, being able to characterize in situ a fully-integrated and deployed transponder is of interest.

To illustrate how such measurements are possible, a scenario of conducting over-the-air laboratory measurements was conducted on an example 2H transponder over a varying azimuth angle (ϕ). As illustrated in FIG. 17, a harmonic transponder 1700 was placed on rotating platform (not shown) and interrogated with an interrogator 1704 using a dual-frequency interrogation signal 1708. The interrogator 1704 also included instrumentation for measuring various characteristics of a return signal 1712 backscattered from the transponder 1700. With the EIRP of the interrogation signal 1708 fixed throughout the test, the transponder 1700 was rotated to different positions, as would be done with any antenna azimuth pattern test. The EIRP used in this testing corresponds to −27 dBm at the transponder 1700, when normally oriented (i.e., ø=0°). That is, the test conditions leading the response shown in FIG. 4.

As the transponder 1700 is rotated away from its normal orientation, two things will occur. First, the absolute power of the return signal 1712 that the interrogator 1704 receives can be expected to change. This will be due to (i) changing gain from the receive antenna (not shown) of the transponder 1700 (G_{ht,f}) as a function of angle, causing (ii) a change in the power incident at the transponder's diode (not shown), thereby causing a change in the diode's conversion (CL_{d}), and (iii) changing gain from the device's transmit antenna (G_{ht,2f}).

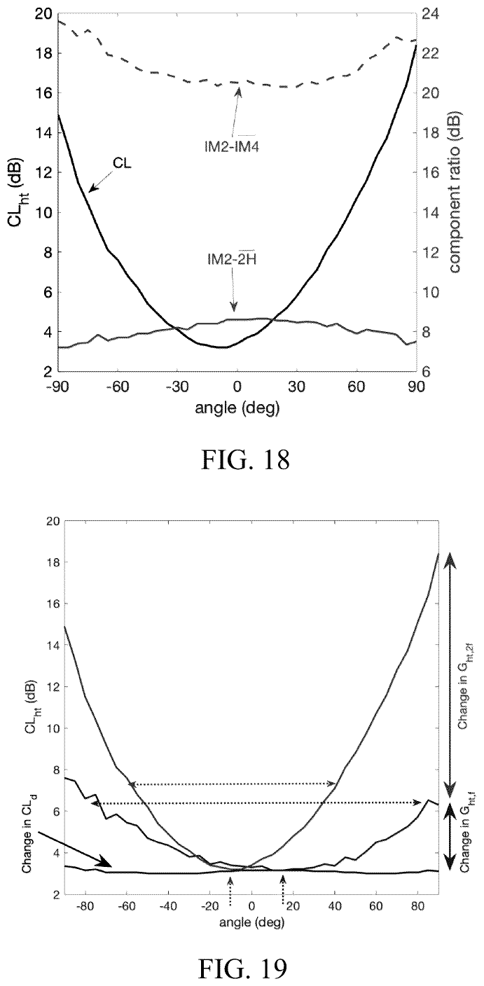

Second, changing the incident power at the diode of the transponder 1700 causes its nonlinear characteristics to change, which impacts the measurable IMD ratios. Such data is shown in FIG. 18. The lefthand y-axis shows the single frequency conversion loss. As would be expected, as the transponder 1700 is rotated from the receive and transmit peak-of-beams orientation, the conversion loss increases relative to the conversion loss for its normal orientation.

The right y-axis shows the IM2- [symbol] and IM2-IM̅4 ratios over the azimuth angle, ϕ, indicating decreasing and increasing values, respectively, over angle. These data, along with FIGS. 6A and 6B discussed above, can be used to determine the change in OP relative to ϕ=0°. Any variation in IMD ratios from the baseline measurement (i.e., at angle 0°), will only be due to a change in the receive antenna pattern, G_{ht,f}, of the transponder 1700 (FIG. 17). Any change in the diode's conversion loss can be found from knowing this change in the effective OP. FIG. 19 plots these individual changes. It is noted that the G_{ht,f} degrades ˜3 dB to above 4 dB over the +90° azimuth sweep. This variation increases the total conversion loss ˜0.5 dB, but only at the edge of the pattern.

Any remaining change in the total conversion loss is due solely to changes in the transponder's transmit antenna pattern (G_{ht,2f}) with angle, which can be calculated using Equation 3, below. From the testing described in this section, this change in gain is also shown in FIG. 19.

$${G}_{ht , 2 f} = {\underbrace{{P}_{I , 2 f}}}_{measured} + {\underbrace{{G}_{I , 2 f} - {L}_{ref , 2 f}}}_{known} + {\underbrace{OP}}_{IMD data} - {\underbrace{{CL}_{d}}}_{CL data} \qquad ( 7 )$$

Marked on FIG. 19 are both the pattern center points and 3 dB beamwidths. Comparing to simulation results of just the individual transponder antennas, both patterns should have boresights at 0°. From the present testing data, slight shifts between the receive and transmit patterns can be seen. The 3 dB beamwidth for the receive antenna is ˜155°, versus 180° provided by simulation. Similarly, the present testing measured ˜100° for the 3 dB transmit beamwidth, slightly narrower that the ˜113° noted in the simulation. While these results are not exact to what were simulated, they represent data for the fully integrated transponder 1700 and illustrate performance that is not unexpected for the given design. In short, using remote measurements, the individual contributions of the transponder's three distinct components to the overall device's conversion loss can be deconstructed using the IMD-ratio approach.

Like any over-the-air measurement, the uncertainty associated with a single power measurement is non-zero. Based on the data presented in FIG. 19, OP data from the two different IMD-ratio measurements (i.e., IM2-2H and IM2-IM4) are consistently within +1 dB of each other over the full range of azimuth measurements made. This uncertainty is well within that reported for other over-the-air measurement campaigns.

This example illustrates that, by using this method, the signal changes at the interrogation frequency, f, can be isolated separately from the signal changes at the backscattered frequency, 2f, while also accounting for any nonlinearity produced by the diode. This demonstration is a surrogate for showing that if a harmonic transponder were embedded within an unknown media, then the approach could be used to determine channel losses at f and at 2f. Having two loss measurements at two different frequencies for the same channel, could be advantageous for applications, such as sensing moisture in soil, snow, or other medium with a buried harmonic transponder.

### III. Example Methods

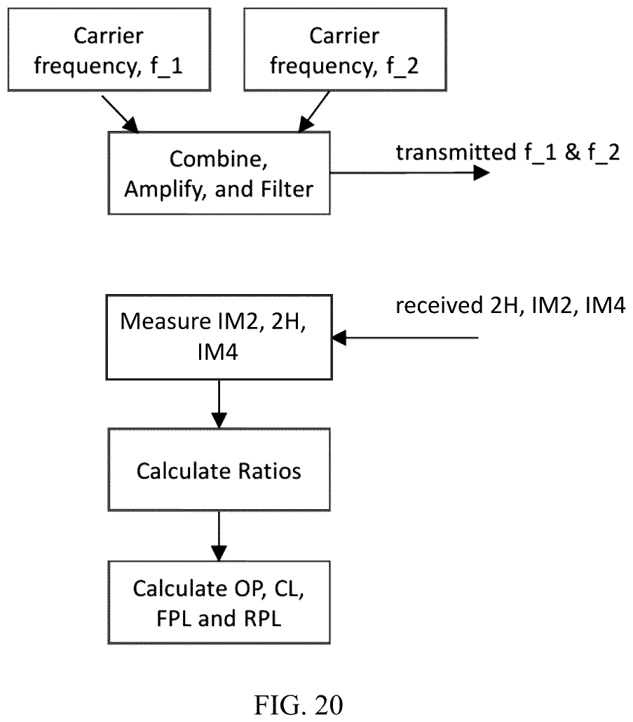

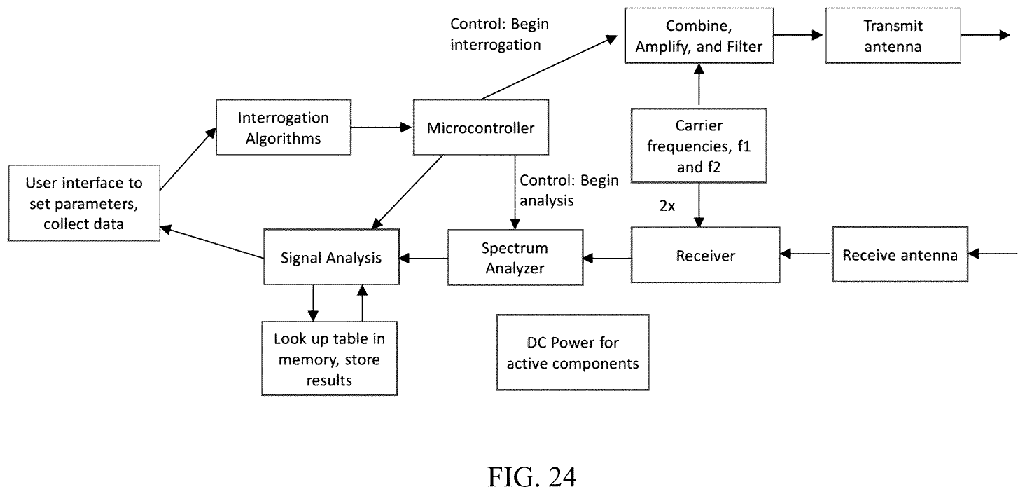

FIG. 20 illustrates an example method that can be performed, for example, by an interrogator and/or other system (not shown, but see, for example, accompanying FIGS. 24 through 26) of the present disclosure. The upper three blocks of FIG. 20 illustrate generating an interrogation signal that contains two closely spaced frequencies, f_1 and f_2 and that is targeted at a 2H transponder (not shown). The lower three blocks of FIG. 20 illustrate the processing of a return signal backscattered from the 2H transponder that contains 2H, IM2, and IM4 components so as to determine values for one or more of the OP, the CL, the forward power loss (FPL), and the returned power loss (RPL). Examples of how the various blocks of FIG. 20 can be performed are discussed in other parts of this disclosure. Those skilled in the art will readily appreciate how to modify the method of FIG. 20, if needed, without undue experimentation and using the present disclose as a guide.

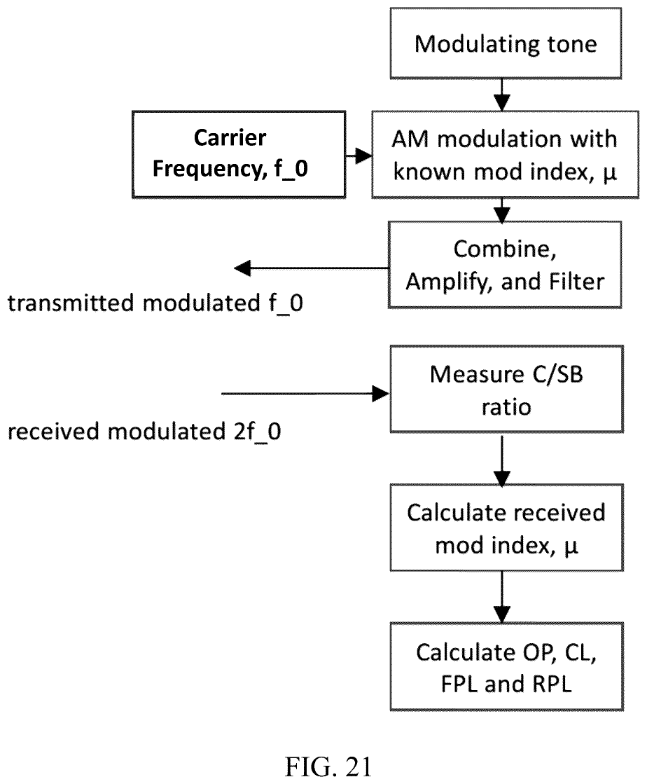

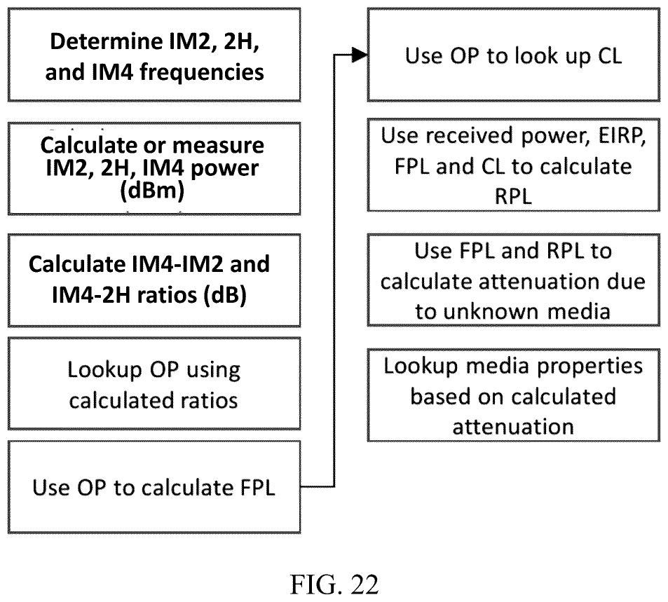

FIG. 21 illustrates an example method that can be performed, for example, by an interrogator and/or other system (not shown, but see, for example, accompanying FIGS. 24 through 26) of the present disclosure. The upper four blocks of FIG. 21 illustrate generating an interrogation signal (transmitted modulated f₀) that contains an AM signal modulated with a modulating tone and that is targeted at a 2H transponder (not shown). The lower three blocks of FIG. 21 illustrate the processing of a return signal (received modulated 2f₀) backscattered from the 2H transponder that so as to determine values for μ and one or more of the OP, the CL, the FPL, and the RPL. Examples of how the various blocks of FIG. 21 can be performed are discussed in other parts of this disclosure. Those skilled in the art will readily appreciate how to modify the method of FIG. 21, if needed, without undue experimentation and using the present disclose as a guide.

FIG. 22 illustrates an example method that can be performed, for example, by an interrogator and/or other system (not shown, but see, for example, accompanying FIGS. 24 through 26) of the present disclosure. In this example, the method uses a process involving interrogating a 2H transponder (not shown) embedded or otherwise located in a medium using a dual-spaced-apart-frequency interrogation signal, determining various characteristics of a return signal that the 2H transponder backscatters, and then using the characteristics to lookup one or more properties of the medium in which the 2H transponder is embedded or located in. Examples of how the various blocks of FIG. 22 can be performed are discussed in other parts of this disclosure. Those skilled in the art will readily appreciate how to modify the method of FIG. 22, if needed, without undue experimentation and using the present disclose as a guide.

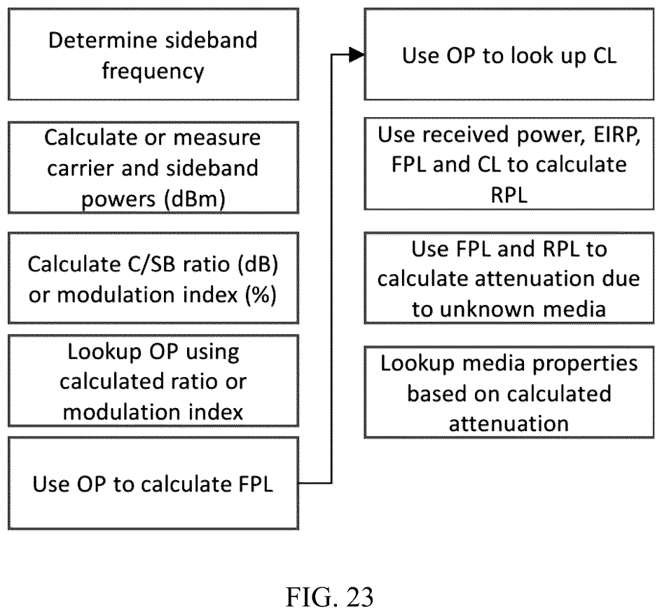

FIG. 23 illustrates an example method that can be performed, for example, by an interrogator and/or other system (not shown, but see, for example, accompanying FIGS. 24 through 26) of the present disclosure. In this example, the method uses a process involving interrogating a 2H transponder (not shown) embedded or otherwise located in a medium using a tone-modulated AM interrogation signal, determining various characteristics of a return signal that the 2H transponder backscatters, and then using the characteristics to lookup one or more properties of the medium in which the 2H transponder is embedded or located in. Examples of how the various blocks of FIG. 23 can be performed are discussed in other parts of this disclosure. Those skilled in the art will readily appreciate how to modify the method of FIG. 23, if needed, without undue experimentation and using the present disclose as a guide.

### IV. Example Interrogators

FIG. 24 illustrates an example interrogator or other system that is configured to perform, for example, a process involving interrogating a 2H transponder (not shown) embedded or otherwise located in a medium using a dual-spaced-apart-frequency interrogation signal, determining various characteristics of a return signal that the 2H transponder backscatters, and then using the characteristics to lookup one or more properties of the medium in which the 2H transponder is embedded or located in or any other processed disclosed herein. Examples of how the various components illustrated in FIG. 24 can be implemented will be apparent to those skilled in the art from reading this entire disclosure and using only ordinary skill in the art. Those skilled in the art will readily appreciate how to modify the transponder/system of FIG. 24, if needed, without undue experimentation and using the present disclose as a guide.

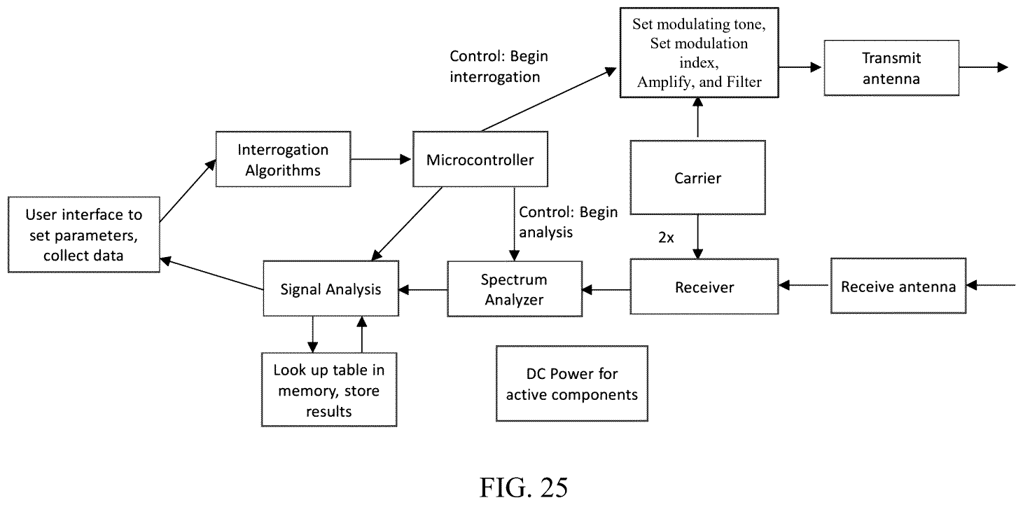

FIG. 25 illustrates an example interrogator or other system that is configured to perform, for example, a process involving interrogating a 2H transponder (not shown) embedded or otherwise located in a medium using a tone-modulated AM interrogation signal, determining various characteristics of a return signal that the 2H transponder backscatters, and then using the characteristics to lookup one or more properties of the medium in which the 2H transponder is embedded or located in or any other processed disclosed herein. Examples of how the various components illustrated in FIG. 25 can be implemented will be apparent to those skilled in the art from reading this entire disclosure and using only ordinary skill in the art. Those skilled in the art will readily appreciate how to modify the transponder/system of FIG. 25, if needed, without undue experimentation and using the present disclose as a guide.

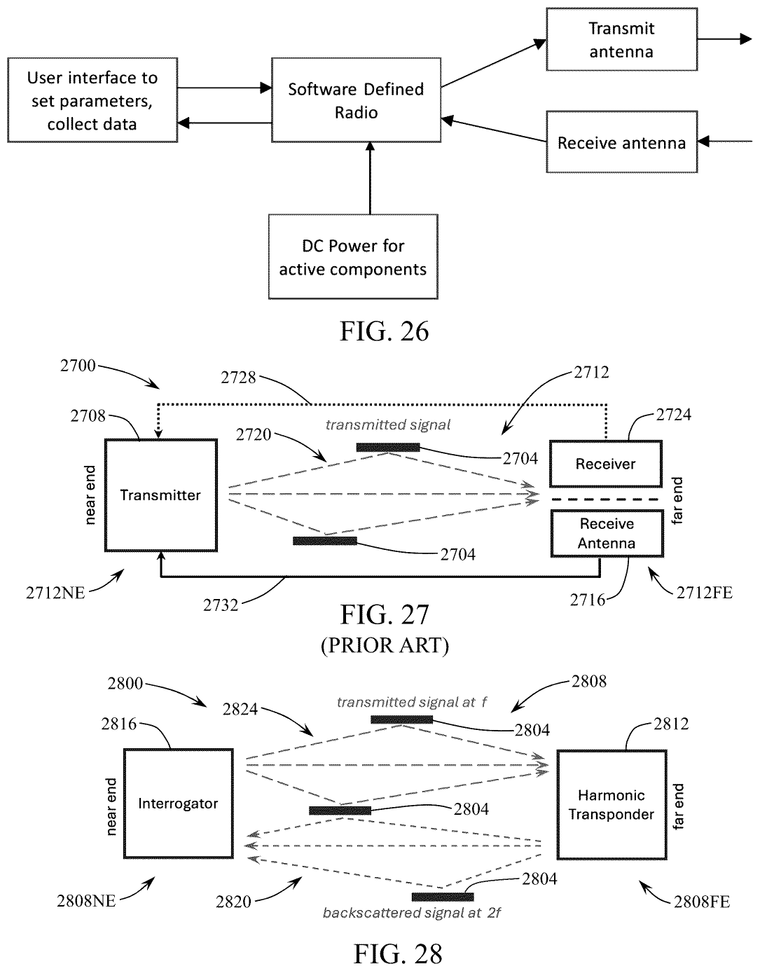

FIG. 26 illustrates an example software defined radio system that can be used in an interrogator or other system of the present disclosure, such as either of the interrogators/systems shown in FIGS. 24 and 25. Examples of how the various components illustrated in FIG. 26 can be implemented will be apparent to those skilled in the art from reading this entire disclosure and using only ordinary skill in the art. Those skilled in the art will readily appreciate how to modify the software defined radio system of FIG. 26, if needed, without undue experimentation and using the present disclose as a guide.

### V. Wireless Channel Characterization

Modern wireless communication systems are operating in ever more complex environments, particularly with the advent of the “Internet of things” (IoT) composed of low-power and/or low energy devices. Built environments, such as factories, ships, and aircraft, among many others, are prone to introducing significant multipath due to metallic structures within these environments. Knowing the extent of multipath is critical when designing a wireless system, particularly when the power and/or energy of the communicating devices is limited.

Multipath characterization of wireless communication channels is a well-studied field. Empirical methods, referred to broadly as “channel sounding”, for a multipath environment 2700 containing one or more signal reflectors (e.g., metal objects), here, two signal reflectors 2704, require setups comparable to that shown in FIG. 27, in which a transmitter/transmitting antenna 2708 is at the near end 2712NE end of a wireless link 2712 and a receive antenna 2716 is at the far end 2712FE of the wireless link and the transmitter transmits a sounding signal 2720. The antennas 2708 and 2716 can be connected to separate transmitters, such as transmitter 2708 and receivers, such as receiver 2724, that are synchronized (dotted line 2728) or cabled to a single unit (solid line 2732), for example, a vector network analyzer (VNA). The sounding signal can be short pulse, which in a multipath environment, such as the multipath environment 2700 will be received as multiple pulses. Alternatively, the sounding signal can be a frequency sweep, and the received signal will measure the channel frequency response (CFR). The sounding signal can be a continuous wave (CW) signal, where the power at the receiver is monitored as the transmitter and/or receiver are spatially moved. In all three scenarios, the measured spatial-fading data can be used to classify the wireless link 2712, a/k/a “channel”, as Rician, Rayleigh, or potentially worse-than-Rayleigh.

Regardless of the approach, at the far end of the wireless link there needs to be either a dedicated receiver or an antenna that is cabled to the transmitter at the near end. For very cluttered environments, the placement of this instrumentation and/or cabling may be challenging. Other environments could be harsh and thus potentially damaging to the equipment or could be hazardous and, thus, be dangerous for those placing this instrumentation.

This section presents novel alternatives that leverage compact, low-cost, passive wireless nonlinear frequency-multiplying transponders, such as harmonic transponders, to characterize multipath by measuring each of three differing types of fading, namely, spatially-dependent fading, frequency-dependent fading, and time-dependent fading. As illustrated in FIG. 28, for each of these fading types in a multipath environment 2800 having one or more signal reflectors 2804 (e.g., metal objects) over a wireless link 2808, a wireless harmonic transponder 2812 is placed at the far end 2808FE of the wireless link, while an interrogator 2816, which combines transmitter and receiver functionality, is placed at the near end 2808NE. Because the transponder 2812 backscatters a harmonic 2820 of the interrogation signal 2824, the forward link (near to far for the interrogation signal) can be characterized separately from the reverse link (far to near for the harmonic). Specific example techniques for characterizing multipath by measuring spatially-dependent fading, frequency-dependent fading, and time-dependent fading are presented, respectively, in the following three subsections.

Wireless systems deployed in either static or dynamic, cluttered environments are prone to experiencing multipath, which can result in significant weakening of signal strength (or fades) that occurs as a function of frequency, space, and/or time. The disclosed methods provide a means to measure the multipath caused by the environment using instrumentation located only at one side of a communication link, as noted above. Known multipath characterization methods can then be applied to these measured data. Based on the characterization, users may then employ a variety of known multipath mitigation methods.

For example, for frequency-selective or spatially-selective fading, users may employ known methods such as channel diversity and/or antenna diversity. As another example, for time-selective fading, the communication system may utilize known modulation methods such as code division multiple access (CDMA), along with a rake receiver, to mitigate time-varying fades. Implementing these and/or other mitigation methods ensures the wireless communication link remains reliable, even as the environment it is deployed in changes. The disclosed methods make it possible to collect the necessary characterization data even if the environment is too cluttered or hazardous for known measurement approaches.

### V.A Spatial-Dependent Fading

This example uses constant-tone AM as discussed above in detail in section 1.D. As described there, the modulation index, μ, manifests itself in the frequency domain as a carrier-to-sideband ratio (C/SB) between the power in the carrier frequency, at f_{c}, and the two sidebands, at f_{c}−fₘ and f_{c}+fₘ. The C/SB ratio is minimized for μ=100%, where each sideband has one quarter the power of the carrier, giving a C/SB of 6 dB. As μ decreases, C/SB increases. For example, for 50%, the theoretical C/SB is 12 dB. In a linear channel, the C/SB of the transmitted signal will be preserved at the receiver. However, for the bidirectional link of FIG. 28, with a nonlinearity introduced by the harmonic transponder, the C/SB is not always preserved, and this attribute is exploited for channel multipath characterization.

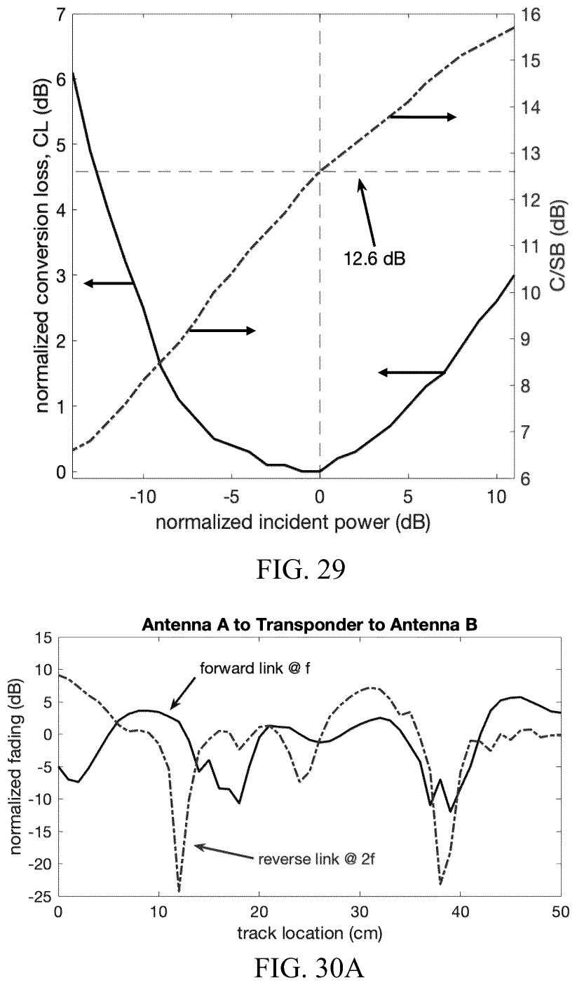

Referring to FIG. 29 and also to FIG. 28, the graph in FIG. 29 (right y-axis) shows how the C/SB of the backscattered signal 2820 (FIG. 28) is dependent on the power incident at the harmonic transponder 2812. In these data, the C/SB of the interrogator 2816 was 12.6 dB. At the power that minimizes the conversion loss, i.e., at

$${P}_{ht , f}^{↓} ,$$

we see that the backscattered C/SB is the same. However, as the incident power increases beyond

$${P}_{ht , f}^{↓} ,$$

the backscattered C/SB ratio increases. Conversely, the C/SB ratio decreases as the incident power drops. In short, by measuring the C/SB of the backscattered signal 2820 received at the near end 2808NE of the wireless link 2808, the power incident at the harmonic transponder 2812 located at far end can be remotely determined.

### V.A.1 Determination of Pathloss

With continuing reference to FIG. 28, this section describes a means to isolate and characterize the multipath in the forward link (interrogation signal 2824) separately from that on the reverse link (harmonic 2820). First, the budget of the forward link is investigated.

The power incident at the harmonic transponder 2812, P_{ht,f}, is directly proportional to the EIRP (again, Effective Isotropic Radiated Power) of the interrogator 2816 and the forward path loss (FPL_{f}), as illustrated in Equation 8, below (all equations in the subsection utilize logarithmic values, i.e., dB, dBi, and dBm). However, as multipath is a small-scale fading effect, the path loss is not evident. Furthermore, as the harmonic transponder 2812 is remote from the interrogator instrumentation, P_{ht,f} cannot be directly measured.

$${\underbrace{{P}_{ht , f}}}_{?} = {\underbrace{{P}_{tx , f} + {G}_{tx , f}}}_{known , EIRP} + {\underbrace{{FPL}_{f}}}_{?} , \qquad ( 8 )$$

However, as illustrated in FIG. 27, using an AM interrogation signal, the backscattered C/SB can be monitored to ascertain the power at the device. By adjusting the transmitted power, the EIRP can be set to the value that matches the transmitted and received C/SB, i.e., the power of minimum conversion loss

$${P}_{ht , f}^{↓} ,$$

thereby allowing us to determine the FPL_{f} as follows.

$${FPL}_{f} = {\underbrace{{P}_{tx , f} + {G}_{tx , f}}}_{EIRP adjusted} - {\underbrace{{P}_{ht , f}^{↓}}}_{found by C / SB} \qquad ( 9 )$$

As the multipath conditions change, the FPL_{f} will change, but these changes can be tracked by readjusting the EIRP to achieve

$${P}_{ht , f}^{↓}$$

at the device.

To find the reverse link path loss (RPL_{2f}), the process starts with its link equation, Equation 10, below. Here the gain of the receive antenna (G_{rx,2f}) of the interrogator 2816 is known, and the received backscattered power at the receiver, P_{rx,2f}, of the interrogator is measured. As the harmonic transponder 2812 is a nonlinear device, its backscattered power, P_{ht,2f}, is not generally known.

$${\underbrace{{P}_{rx , 2 f}}}_{measured} = {\underbrace{{P}_{h t , 2 f}}}_{?} - {\underbrace{RP {L}_{2 f}}}_{?} + {\underbrace{{G}_{rx , 2 f}}}_{known} \qquad ( 10 )$$

However, by assuring the power at the harmonic transponder 2812 is at

$${P}_{ht , f}^{↓} ,$$

the reradiated power is known to be

$${P}_{h t , 2 f}^{↑} \text{?}$$

$$\text{?} \text{indicates text missing or illegible when filed}$$

through the following expression, wherein CLₘᵢₙ is the minimum conversion loss of the harmonic transponder.

$${P}_{h t , 2 f}^{↑} = {P}_{ht , f}^{↓} - {CL}_{min} \qquad ( 11 )$$

Note that the performance (i.e.,

$${P}_{h t , f}^{↓}$$

and CLₘᵢₙ) of the harmonic transponder 2812 can readily be measured, prior to deployment using the typical unmodulated interrogation approach. Under the conditions wherein the device is receiving

$${P}_{h t , f}^{↓} ,$$

the RFL_{2f} can then be found as follows.

$${RPL}_{2 f} = {\underbrace{{P}_{ht , 2 f}^{↑}}}_{Eq . ( 11 )} + {\underbrace{{G}_{rx , 2 f}}}_{known} - {\underbrace{{P}_{rx , 2 f}}}_{measured} \qquad ( 12 )$$

Once the transmitted EIRP has been adjusted to achieve

$${P}_{ht , f}^{↓} ,$$

any change in the measured received power corresponds directly to the change in the channel loss (i.e., multipath). In summary, both the FPL_{f} and the RPL_{2f} can be found using the following procedure.
1. Configure the interrogation signal for a known C/SB_{tx,f}.
2. Adjust the EIRP until the backscattered CSB_{rx,2f}, as measured at the receiver, equals C/SB_{tx,f}.
3. Record transmitted and received powers (i.e., P_{tx,f} and P_{rx,2f}, respectively).
4. Use Equations (9) and (12), above, to calculate FPL_{f} and RPL_{2f}, respectively.

### V.A.2 Results

This section demonstrates the presented methodology in which a multipath, experienced by a harmonic transponder, through a reflective environment was measured and characterized using the methodology disclosed above in this section.

### V.A.2.a Test Setup and Environment

The test setup consisted of an interrogator and harmonic transponder configured as illustrated in FIG. 28. An RF signal generator (SG, Rigol, DSG815) was used to send a signal at 890 MHz. This signal was amplitude modulated using a 10 kHz tone and with a modulation index of μ=50%. The measured C/SB at this source was 12.6 dB (vs. theoretical 12 dB). This signal was amplified (RF Lambda, RFLUPA02M03G2), and low pass filters (Mini Circuits, VLF-1200) were used to ensure any harmonics appearing at the amplifier output were suppressed. This interrogation signal was sent using a 15 cm monopole antenna. The backscattered signal, at 1.78 GHz, was received with an identical, but separate antenna. This received signal was high pass filtered (Mini Circuits, VHF-1500) to suppress any leakage at 890 MHz. A spectrum analyzer (SA, Anritsu, MS2036A) was used to analyze the signal's received power and C/SB.

The test environment was a 91.4 cm×30.5 cm×91.4 cm compact reverberation chamber designed to create a variety of multipath conditions ranging from benign Rician to severe hyper-Rayleigh conditions. For the interrogation frequency of 890 MHz, the chamber's maximum dimension is only ˜3λ, notably smaller than chambers designed to create statistically uniform environments for EMC/EMI testing. The harmonic transponder was placed on a 50 cm linear track, which allowed it to be moved in 1 cm increments. At each of the resulting 51 locations (across ˜1.5 λ at the interrogation frequency, f), two data values were recorded as follows.
1) Transmitting from antenna A, the signal generator's output power was adjusted in 0.1 dB increments until the C/SB_{rx,2f}=C/SB_{tx,f}, i.e., until the transponder's received power was

$${P}_{ht , f}^{↓} .$$

Then, the signal generator's output power was recorded.
2) The power received at antenna B was then recorded.

### V.A.2.b Within-Chamber Channel Response

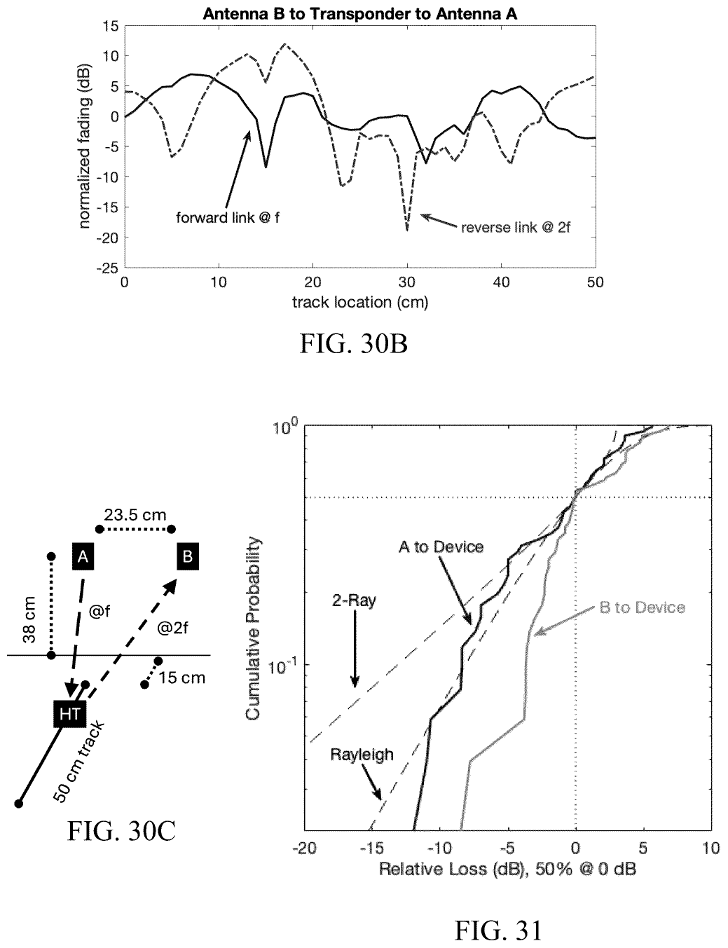

After collecting these data for all track positions, the values of both the transmit powers and the received powers were normalized to their respective medians. In addition, the sign of the normalized transmit powers was changed, as an increase in transmitted power indicates that fading has increased. The resulting data for the forward and reverse links can be seen in FIG. 30A, with the forward link at frequency f being shown by the solid line and the reverse link at frequency 2f being shown by the dashed line. Viewing FIG. 30A, it is seen that the forward link (solid line) exhibited a narrower range of facing than the reverse link (dashed line). This matches the expectations that multipath gets more severe with frequency. Two distinct nulls are present in the reverse link (dashed line). This corresponds to a change in the harmonic transponder (i.e., “device”) to receive antenna distance of ˜1λ. In short, the present methodology produced data that would be not unexpected for the chamber environment of the test setup.

The process was repeated, but with the transmit and receive antennas swapped (i.e., transmitting with antenna B, receiving with antenna A). These data are shown in FIG. 30B. Thus the utilized test setup yielded data for two different links, each at two different frequencies. Considering only the forward links, i.e., from the transmit antennas to the harmonic transponder at the frequency of f=890 MHz, their fading statistics are presented in FIG. 31. It is noted that one link (link A to device) exhibited near Rayleigh statistics, while the other (link B to device) was more benign (i.e., Rician K-factor of ˜3 dB).

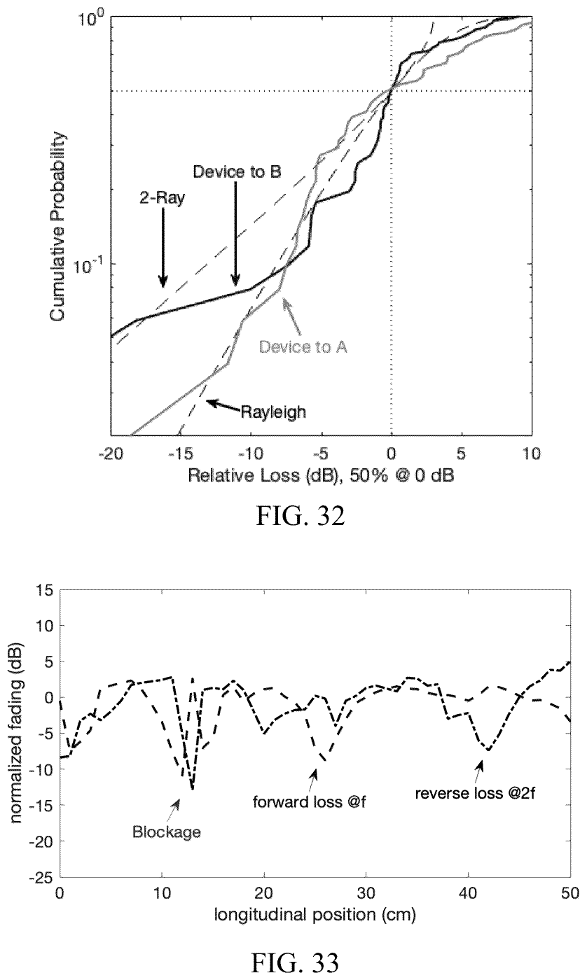

The statistics for the reverse links, i.e., from the device to the receive antennas at the frequency of 2f=1.78 GHz, are shown in FIG. 32 to exhibit more severe fading than the forward links. For instance, it is seen that fades of >15 dB (relative to the median) occur and that the cumulative distribution function approaches the worse-than-Rayleigh two-ray condition.

TABLE I

| | |
|---|---|
| ρ | FIG. |

Frequency independence

| | | |
|---|---|---|
| Path: A ↔ device | −0.15 | 30A |
| Path: B ↔ device | −0.22 | 30B |

Path independence

| | | |
|---|---|---|
| A vs. B to device at 890 MHz | −0.13 | 31 |
| Device to A vs. to B at 1.78 GHz | −0.21 | 32 |

Using the results shown in FIGS. 30A-32, the correlation coefficient (ρ) was calculated for the multipath conditions for (i) the same paths, but at different frequencies, and (ii) the same frequencies, but for different paths. These results are presented above in Table I. These correlation coefficients indicate that all links measured are nearly statistically independent (i.e., |ρ|<˜0.2) of each other.

### V.A.2.c Outside to Inside Chamber Link Measurement

The testing of the preceding subsection utilized links within the compact reverberation chamber to show that the presented method can characterize a wide range of severely fading channels. These links were necessarily short, which raises the question, “what is the maximum distance for which this method can be used?” To address this question, a second test was conducted in which the interrogator was placed outside the chamber at a distance of 2 m. The harmonic transponder, still inside the chamber, was moved laterally across 50 cm, keeping the interrogator/transponder nearly constant.

Forward and reverse channel loss measurements were made, as before, and are presented in FIG. 33. With the exception of a single blockage, there was always line-of-sight and thus these links exhibited more benign fading (Rician K-factors of ˜0 dB). For these measurements, the median transmitted power was ˜25 dBm and the median received power was ˜−60 dBm. Based on these numbers, this approach could be readily implemented at distances of ˜10 m, assuming a 10 W amplifier was used (i.e., ˜15 dB increase) and if the receiver could measure powers down to ˜−110 dBm (30 dB additional path loss+12 dB for the C/SB measurement+10 dB for fades). Even greater distances can be achieved using higher transmit powers and/or lower reference C/SB values.

The results of the testing characterized spatially varying multipath by using a single frequency pair. The method can be extended to multiple frequency pairs throughout the operational bandwidth of the harmonic transponder (e.g., ˜20 MHz for the device utilized), with the result being akin to the CFR. This would allow environments to be characterized, even if the harmonic transponder and interrogator are in fixed positions.

### V.B Frequency-Dependent Fading

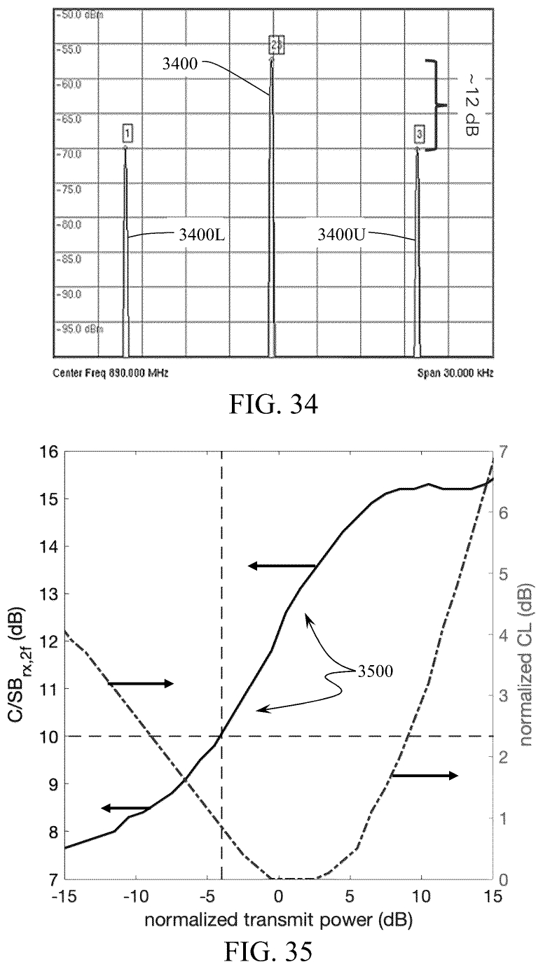

This example also uses constant-tone AM as discussed above in detail in section 1.D. As discussed there, the spectrum of the interrogation signal consists of a carrier and two sidebands, such as shown in FIG. 34 as the carrier 3400, a lower sideband 3400L, and an upper sideband 3400U. In this example, with an AM modulation index, μ, of 50%, the resulting carrier to sideband ratio (C/SB_{tx,f}) of the interrogation signal is fixed at ˜12 dB.

### V.B.1 Determination of Pathloss

Due to the non-linearity of the harmonic transponder 2812 (FIG. 28), the carrier-to-sideband of the backscattered signal (C/SB_{rx,2f}) is dependent on the power incident at the harmonic transponder. That is, as the transmitted power (i.e., EIRP) changes, the (C/SB_{rx,2f} measured in the backscattered signal also changes. This relationship, for a test instantiation of the harmonic transponder 2812 (FIG. 28), is provided using the lefthand axis of the graph of FIG. 35. In FIG. 35, it can be seen that this relationship is nearly linear across a 10 dB power range and that there is an incident power at which C/SB_{rx,2f}=C/SB_{tx,f}=˜12 dB. As discussed above, this incident power is denoted as

$${P}_{ht , f}^{↓} ,$$

and the x-axis of FIG. 35 is normalized to this value.

$${P}_{ht , f}^{↓}$$

also indicates the lowest incident power that minimizes the conversion loss (CL) of the harmonic transponder, that is, the difference between the incident and backscattered powers. The righthand axis of FIG. 35 provides values for the CL curve for the same harmonic transponder 2812 (FIG. 28).

The relationship between a transponder's incident power and its conversion loss can be determined using an over-the-air approach in a calibrated anechoic environment. CLₘᵢₙ will be the minimum difference between

$${P}_{h t , f}^{↓}$$

and the corresponding backscatter power

$${P}_{h t , 2 f}^{↑} \text{?}$$

$$\text{?} \text{indicates text missing or illegible when filed}$$

and is as defined in Equation 11 in the previous subsection above.

For example, the harmonic transponder used in this example has a minimum conversion loss of 3 dB, when the incident power is −25 dBm and which results in backscattered power of −28 dBm. With these incidents and backscattered powers known, calculating the forward (FP_{Lf}) and reverse (RPL_{2f}) path losses is straightforward, as seen, respectively, in Equations 9 and 12 in the previous subsection above.

When the objective is to understand fading, that is, how the channel response changes as a function of, e.g., frequency, one needs only relative measurements. As such, a different target (C/SB_{rx,2f} can be chosen. For example, for the harmonic transponder used in the subject testing, the target received C/SB could be any value in the “linear” region 3500 seen in FIG. 35, i.e., from about 9 dB to about 15 dB. In the subject testing, a target received C/SB of 10 dB. This value has benefits of being reached at lower transmit powers, in comparison to

$${P}_{ht , f}^{↓} ,$$

thus reducing the likelihood of the interrogation signal being distorted by the transmit amplifier of the interrogator 2816 (FIG. 28).

In summary, fading on the forward/interrogation and reverse/backscattered links can be found using the following procedure.
1. Configure the AM interrogation signal for a known C/SB_{tx,f}, e.g., by setting μ=50%.
2. Adjust the EIRP, i.e., P_{tx,f}, until the backscattered CSB_{rx,2f}, as measured at the receiver, matches the target value.
3. Record transmitted and received powers (i.e., P_{tx,f} and P_{rx,2f}, respectively).
4. Change the interrogation frequency and repeat the above Steps (2) and (3).

In addition to the harmonic transponder 2812 (FIG. 28) being nonlinear, its performance is also frequency dependent. As such, to understand the channel's response, the response of the harmonic transponder 2812 needs to be first accounted for. The next section, below, details this methodology fully through an empirical example.

### V.B.2 Results

This section demonstrates the presented methodology by measuring and characterizing the frequency selective channels experienced by a harmonic transponder deployed in a highly reflective environment.

### V.B.2.a Test Setup and Environment

The test setup consisted of an interrogator and a harmonic transponder configured as illustrated in FIG. 28. An RF signal generator (Rigol, DSG815) was used to send a signal between 875 MHz and 900 MHz. This signal was amplitude modulated using a 10 kHz tone and with a modulation index of μ=50%. The nominal measured C/SB at this source was 12.2 dB (vs. theoretical 12 dB). This signal was amplified (RF Lambda, RFLUPA02M03G2), and low pass filters (Mini Circuits, VLF-1200) were used to ensure any harmonics appearing at the amplifier output were suppressed. This interrogation signal was sent using a 15 cm monopole antenna. The backscattered signal, at the second harmonic, was received with an identical, but separate antenna. This received signal was high pass filtered (Mini Circuits, VHF-1500) to suppress any leakage from 890 MHz band. A spectrum analyzer (SA, Anritsu, MS2036A) was used to analyze the signal's received power and C/SB across the 1.75 GHz to 1.80 GHz band.

### V.B.2.b Anechoic Measurements

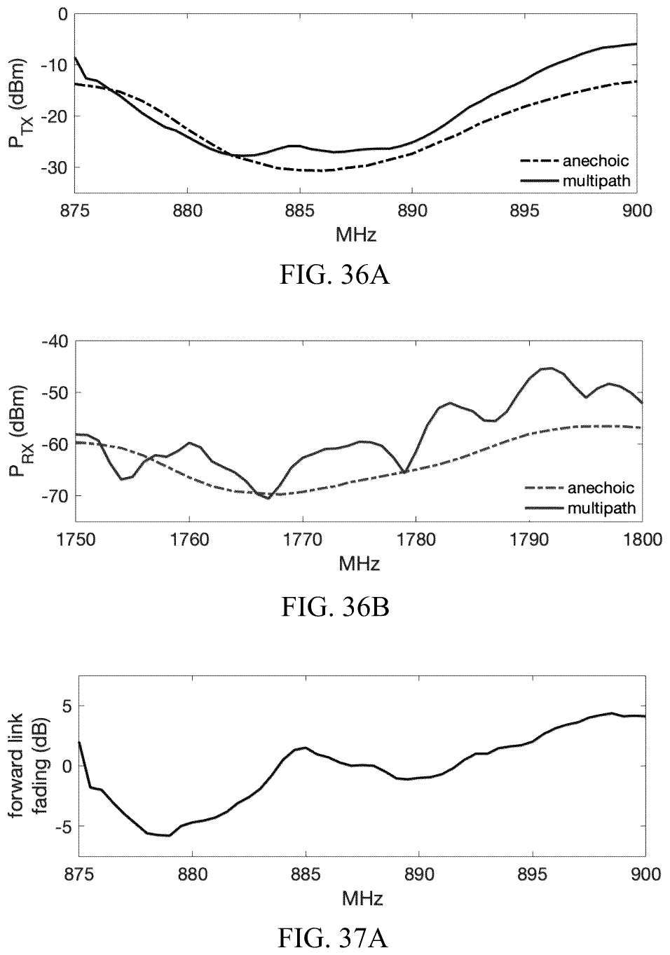

Harmonic transponders, as noted, are frequency-dependent devices and thus have an operational bandwidth. For the device used in this work, that bandwidth is ˜25 MHz centered at ˜888 MHz. To characterize the frequency response of the harmonic transponder, it was placed in an anechoic environment and the four-step procedure provided in section V.B.1 at 51 distinct frequencies between 875 MHz and 900 MHz (i.e., at increments of 500 kHz). These raw transmit and receive power data are presented via the dashed lines in the plots of FIGS. 36A and 36B. From these plots, it is noted that over the 25 MHz measured, the receives response (i.e., across 875 MHz-900 MHz) changes ˜15 dB, while its backscattered response (i.e., across 1.75 GHz-1.80 GHz) of the harmonic transponder changed ˜10 dB. Since the intended purpose is to characterize the channel response, these device effects, now characterized, can be removed.

### V.B.2.c Multipath Environment

To illustrate measuring the CFRs of a multipath environment, a test environment comprising a 90 cm×30 cm×90 cm compact reverberation chamber capable of creating a variety of multipath conditions ranging from benign Rician to severe hyper-Rayleigh conditions was employed. The transmit and receive antennas were placed at the righthand side of the chamber, spaced 23.5 cm apart, and the harmonic transponder was placed on the lefthand side, ˜70 cm away from the transmit and receive antennas. The measurement procedure was repeated and the transmitted and received powers, i.e., P_{tx,f} and P_{rx,2f}, were recorded. These raw data are also shown, respectively, via the solid lines in FIGS. 36A and 36B.

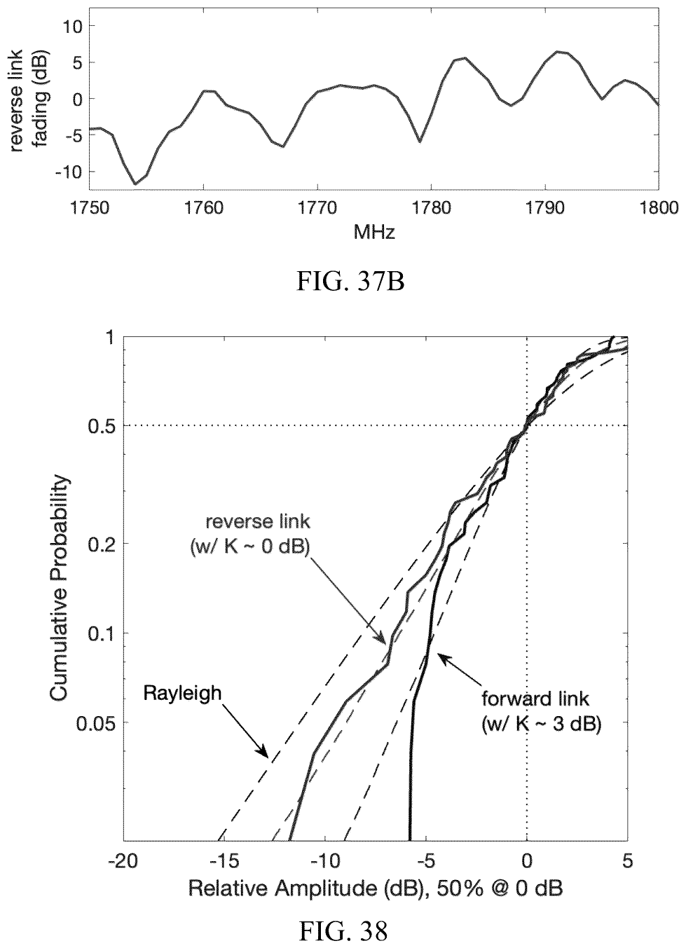

These data include the receive and transmit frequency responses of the harmonic transponder. In FIGS. 37A and 37B, the responses of the harmonic device, found through the anechoic environment test, were removed and the resulting channel frequency responses normalized to the median loss across the measured bandwidth are presented. The fading in the forward link (FIG. 37A) shows less variation than the fading of the reverse link (FIG. 37B). This result would be expected as the wavelength of reverse link signal is half the wavelength of the forward link signal and thus more susceptible to the cluttering in the chamber.

To further characterize these link responses, their cumulative fading probabilities are shown in FIG. 38. Using a 10% fade depth criterion, the forward and reverse link channels were found to exhibit Rician fading with K-factors of ˜3 dB and ˜0 dB, respectively.

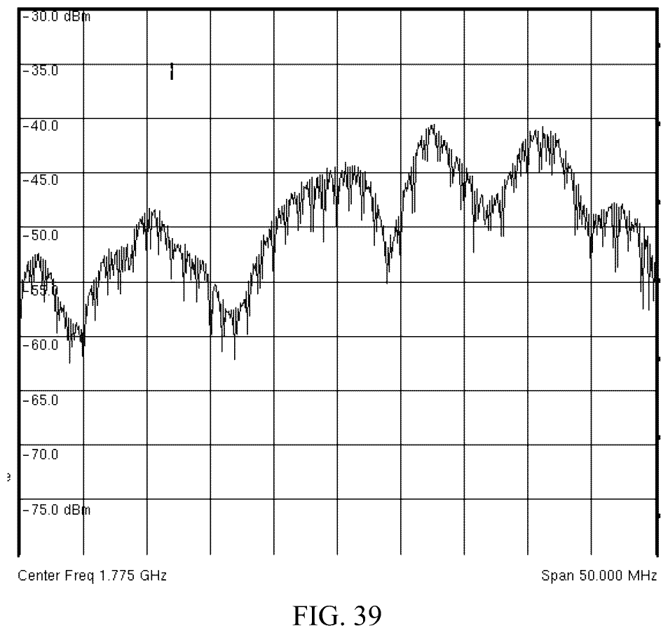

To corroborate the multipath variations seen in the CFRs presented in FIGS. 37A and 37B, the bidirectional response from and to the interrogator were measured using an unmodulated carrier held at a fixed power. The frequency of the signal was swept from 875 MHz to 900 MHz in 100 kHz increments, and the response was measured from 1.75 GHz to 1.80 GHz (i.e., the second harmonic). These results are shown in FIG. 39.

This response not only captures the multipath for both the forward link (at frequency f) and the reverse link (at frequency 2f), but also any changes in the conversion loss CL of the harmonic transponder as its incident power varies. The shape of this response shows the variations seen in FIG. 37B for the backscattered channel, along with the positive trend seen in FIG. 37A for the forward response.

The procedure presented in section V.B.1, shows that this bidirectional response can be decomposed into the CFRs of the forward and reverse links. Any remaining losses observed in these bidirectional data are due to the changes in CL of the harmonic transponder. Because the transmit power was fixed, the incident power at the harmonic transponder varied with the response of the forward link, i.e., ±5 dB from FIG. 37A. From FIG. 35 and the righthand y-axis, the change in incident power of ±5 dB can result in CL changes of several dB. Knowing the extent of these changes depends on knowing where the harmonic transponder is operating (i.e., range of incident power, x-axis in FIG. 35). As indicated throughout this disclosure, knowing the operating point of the harmonic transponder is readily achieved using the AM interrogation approach described herein.

In this example, the frequency selective nature of wireless communication links has been characterized using instrumentation located only at one end of a wireless link and a passive, low-cost harmonic transponder at the other end of the wireless link. The approach allows the CFR to be simultaneously characterized for two links, i.e., the forward and reverse links, using two bands, i.e., the band used to interrogate the transponder and the band at the second harmonic, which the harmonic transponder backscatters. As discussed above, the presented method has applicability for hazardous and/or cluttered environments in which a harmonic transponder can be embedded and where the interrogation system is placed at a safe/remote distance.

Because the harmonic transponders are non-linear devices, with a finite operational bandwidth, it is important to ensure these devices are first “calibrated” in an idealized environment, such as described in this section regarding the anechoic testing. It is noted that this work considered just a single transponder embedded in the environment to be characterized. Because there is no unique identification associated with current harmonic transponder designs, care must be taken so that any additional nearby transponders do not contribute to the measured response.

### V.C Time-Dependent Fading

Sections V.A and V.B, above, are directed to characterizing, respectively, spatially-dependent fading and frequency-dependent fading in multipath environments. Those skilled in the art will readily appreciate that it may also or alternatively be desired to characterize time-dependent fading in certain multipath environments, such as multipath environments having one or more moving objects that reflect wirelessly transmitted signals, such as rotating fans, moving overhead cranes, moving conveyance systems, etc., in which the moving object(s) change(s) the multipath conditions over time.

Those skilled in the art will also readily appreciate that time-dependent fading in such multipath environments can be characterized using either of the methodologies presented above in sections V.A and V.B. In each of these methodologies, having each of the characterizing harmonic transponder and interrogator stationary, the multipath within the dynamic multipath environment is time varying. Thus, either of the above methods can be used over time to determine the statistics, i.e., the characterization, of the dynamic multipath environment. Because the character(s) of the moving object(s) vary from one application to another, the manner in which the multipath-characterization system (i.e., harmonic transponder+interrogator of the present disclosure) is operated for characterization can vary among differing applications. Those skilled in the art will understand how to operate the multipath-characterization system to achieve meaningful multipath characterization statistics based on the nature(s) and character(s) of the relevant moving object(s) present in the dynamic multipath environment under consideration.

Any method disclosed herein, any portion thereof, and any combination of methods disclosed herein and/or otherwise needed to effect any aspect discussed herein may be implemented using and suitable software containing appropriate machine-executable instructions and/or hardware that can process such instructions and/or be controlled based on such instructions. Any machine-executable instructions may be stored in any suitable hardware memory, including, but not limited to RAM of any type, ROM, cache, working memory, long-term storage memory, short-term storage memory, volatile memory, nonvolatile memory, removable memory, etc. For the sake of convenience and custom, any hardware storage memory of any one or more types is referred to herein and in the appended claims as “machine-readable storage medium”, which excludes any transitory storage that can occur on transitory signals, including, digitally encoded carrier signals and pulsed signals encoded with digital data.

The appended claims are incorporated by reference herein in their entireties.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure.

What is claimed is:

1. A method of analyzing an environmental condition, the method comprising:
wirelessly transmitting an interrogation signal that contains first and second frequencies to a nonlinear frequency-multiplying transponder;
receiving a backscattered signal from the nonlinear frequency-multiplying transponder, wherein the backscattered signal contains multiple loss-characterizing components;
measuring features of the multiple loss-characterizing components; and
determining, using the measured features, one or more loss indicia that correlate to environmental losses between the transmitting of the interrogation signal and the receiving of the backscattered signal.

2. The method of claim 1, wherein the environmental condition is a property of a medium in which the nonlinear frequency-multiplying transponder is embedded and the loss indicia correlates to media losses between the transmitting of the interrogation signal and the receiving of the backscattered signal, the method further comprising:
determining, using the loss indicia, a propagation loss of at least one of the interrogation signal and the backscattered signal through the medium; and
determining the physical property of the medium from the propagation loss.

3. The method of claim 2, wherein the medium comprises concrete, and the physical property is moisture contained of the concrete.

4. The method of claim 2, wherein the medium comprises snow, and the physical property is water content of the snow.

5. The method of claim 2, wherein the medium comprises a geotechnic structure, and the physical property is moisture contained in the geotechnic structure.

6. The method of claim 2, wherein the medium comprises soil, and the physical property is soil composition.

7. The method of claim 1, wherein the interrogation signal is an amplitude-modulated signal comprising a carrier signal modulated by a modulating signal, the first frequency being a carrier frequency of the carrier signal and the second frequency being a modulating frequency of the modulating signal, and the multiple loss-characterizing components comprise a multiple of the first frequency and at least one sideband frequency of the multiple of the first frequency.

8. The method of claim 7, wherein the nonlinear frequency-multiplying transponder comprises a harmonic transponder.

9. The method of claim 7, wherein measuring features of the multiple loss-characterizing components includes measuring a carrier-to-sideband ratio.

10. The method of claim 9, wherein the loss indicia comprises an operating point of the nonlinear frequency-multiplying transponder.

11. The method of claim 7, wherein the modulating signal is a constant tone signal.

12. The method of claim 7, wherein the first frequency is in the microwave frequency spectrum.

13. The method of claim 12, wherein the second frequency is in a range of about 1 kHz to about 1 MHz.

14. The method of claim 7, wherein the environmental condition is a multipathing condition and the loss indicia correlates to multipath losses between the transmitting of the interrogation signal and the receiving of the backscattered signal, the method further comprising:
determining, using the loss indicia, a propagation loss of at least one of the interrogation signal and the backscattered signal through the medium; and
determining a multipath fading from the propagation loss, wherein the multipath fading is selected from the group consisting of spatially-dependent fading, frequency-dependent fading, and time-dependent fading.

15. The method of claim 14, wherein the nonlinear frequency-multiplying transponder comprises a harmonic transponder.

16. The method of claim 14, wherein measuring features of the multiple loss-characterizing components includes measuring a carrier-to-sideband ratio.

17. The method of claim 14, wherein the modulating signal is a constant tone signal.

18. The method of claim 14, wherein the multipathing condition exists along a wireless-communication channel within a target environment, wherein the wireless-communication channel is for two or more communications devices located at differing locations within the target environment to wirelessly communicate via communication signals.

19. The method of claim 18, wherein the multipathing condition results from presence, within the target environment, of one or more signal reflectors, one or more signal blockers, or one or more of each of signal reflectors and signal blockers, wherein, when present, each signal reflector and signal blocker interferes with the communication signals as the communication signals travel between the differing locations.

20. A machine-readable storage medium containing machine-executable instructions for performing the method of claim 1.
